SEQUENCE LISTING (8 sequences)

SEQ ID NO: 1 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 2 - protein, 6 aa, Homo sapiens
QIDSPL

SEQ ID NO: 3 - protein, 8 aa, Homo sapiens
TQIDSPLN

SEQ ID NO: 4 - protein, 739 aa, Mus musculus
MPVKMVAVLGASTVLWILFAVSQAFKIEISPEYKTIAQIGDSMALTCSTTGCESPLFSWRTQIDSPLNAKVRTEGSKSVLTMEPVSFENEHSYLCTATCGSGKLERSIHVDIYSFPKDPEIQFSGPLEVGKPVTVKCLAPDIYPVYRLEIDLFKGDQLMNRQEFSSEEMTKSLETKSLEVTFTPVIEDIGKALVCRAKLHIDQIDSTLKERETVKELQVYISPRNTTISVHPSTRLQEGGAVTMTCSSEGLPAPEIFWGRKLDNEVLQLLSGNATLTLIAMRMEDSGVYVCEGVNLIGRDKAEVELVVQEKPFIVDISPGSQVAAQVGDSVVLTCAAIGCDSPSFSWRTQTDSPLNGVVRNEGAKSTLVLSSVGFEDEHSYLCAVTCLQRTLEKRTQVEVYSFPEDPVIKMSGPLVHGRPVTVNCTVPNVYPFDHLEIELLKGETTLMKKYFLEEMGIKSLETKILETTFIPTIEDTGKSLVCLARLHSGEMESEPKQRQSVQPLYVNVAPKETTIWVSPSPILEEGSPVNLTCSSDGIPAPKILWSRQLNNGELQPLSENTTLTFMSTKRDDSGIYVCEGINEAGISRKSVELIIQVSPKDIQLTVFPSKSVKEGDTVIISCTCGNVPETWIILKKKAKTGDMVLKSVDGSYTIRQAQLQDAGIYECESKTEVGSQLRSLTLDVKGKEHNKDYFSPELLALYCASSLVIPAIGMIVYFARKANMKGSYSLVEAQKSKV

SEQ ID NO: 5 - protein, 739 aa, Homo sapiens
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 6 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 7 - protein, 272 aa, Homo sapiens
PEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWK

SEQ ID NO: 8 - protein, 530 aa, Homo sapiens
MPPPRLLFFLLFLTPMEVRPEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWKGGGSGGGSPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0242731 A1
(43) Pub. Date: Aug. 20, 2026

(54) STIMULATORY ANTIGEN PARTICLES, METHODS OF PRODUCTION, AND METHODS OF USE THEREOF

(71) Applicant: Genentech, Inc., South San Francisco, CA (US)

(72) Inventors: Richard Carpenedo, Vancouver (CA); Siddarth Chandrasekaran, Vancouver (CA); Manu Thomas Kalathottukaren, Vancouver (CA); Hio Fan Hoi, Vancouver (CA); Elisa Martinez, Vancouver (CA); Alessia Pallaoro, Vancouver (CA); Steven Woodside, Vancouver (CA)

(21) Appl. No.: 19/418,259

(22) Filed: Dec. 12, 2025

Related U.S. Application Data
(63) Continuation of application No. PCT/CA2024/050792, filed on Jun. 12, 2024.
(60) Provisional application No. 63/472,788, filed on Jun. 13, 2023.

(51) Int. Cl. C12N 5/0783; A61K 35/17; A61K 40/11; A61K 40/31; A61K 40/42; C07K 14/705

(52) U.S. Cl. CPC C12N 5/0636; A61K 35/17; A61K 40/11; A61K 40/31; A61K 40/4211; C07K 14/70503; C12N 2501/42; C12N 2501/599; C12N 2502/1171

(57) ABSTRACT

The present disclosure provides cell stimulatory particles and methods of using same. Also provided are populations of cells produced using the cell stimulatory particles.

### SEQUENCE LISTING

The present application contains a Sequence Listing which has been submitted electronically in 5 XML format named “2011083-0003_SL.XML.” The XML file was created on Dec. 10, 2025 and is 10,940 bytes in size. The entire contents of the Sequence Listing are hereby incorporated by reference.

### FIELD

The present invention generally relates to cell stimulatory particles, and methods of use thereof.

### BACKGROUND OF THE DISCLOSURE

Various feeder cell-based methods are available for the generation of T lineage cells from stem/progenitor cells. However, the expression of cell surface proteins by the supportive stromal cells in feeder cell-based systems is heterogenous, leading to diverse outcomes. Further, feeder cell-based systems are not easily scalable to meet the needs of clinical manufacturing.

Immobilized Notch signalling ligands, such as DL4, in combination with VCAM-1, have been shown to promote in vitro generation of progenitor T cells in a feeder-free and serum-free culture system (Shukla et al., 2017). Microbeads modified to present DL4 have also been shown to support in vitro differentiation of T-lineage cells, albeit with limited progression to mature lineages such as CD4-CD8+ cells (Trotman-Grant et al., 2021). Short-term stimulation of T cell progenitors with a CD3 antibody in the absence of Notch signalling followed by maturation without CD3 antibody has been shown to promote generation of CD8αβ+ T cells (Iriguchi et al., 2021).

Immobilized and soluble antibodies have previously been used to stimulate and expand primary (i.e., donor-derived) T cells, such as through stimulation of a chimeric antigen receptor (CAR) (Philipson et al., 2020). Antigens presented on particles and by antigen-presenting cells have also previously been used to stimulate and expand CAR+ primary T cells (Wu et al. US 20180223255).

It is desirable to obviate or mitigate one or more of the above deficiencies.

### SUMMARY OF THE DISCLOSURE

In an aspect, a method of generating CD4−CD8+ T cells is provided. The method comprises contacting a population of CD4+CD8+ cells expressing an antigen receptor with an antigen immobilized to a substrate. The antigen binds to the antigen receptor.

In an embodiment, the CD4+CD8+ cells are derived in vitro from hematopoietic stem/progenitor cells.

In an embodiment, the hematopoietic stem/progenitor cells are derived in vitro from pluripotent stem cells.

In an embodiment, the CD4+CD8+ cells are derived according to a method comprising contacting a population of hematopoietic stem/progenitor cells with an immobilized Notch signalling ligand.

In an embodiment, the step of contacting the population of hematopoietic stem/progenitor cells with the immobilized Notch signalling ligand further comprises contacting the population of hematopoietic stem/progenitor cells with the immobilized Notch signalling ligand at a first ligand concentration, generating a population of progenitor T cells; and contacting the population of progenitor T cells with the immobilized Notch signalling ligand at a second ligand concentration, lower than the first ligand concentration.

In an embodiment, the first ligand concentration is 3.15×10¹¹ to 1.26×10¹² molecules/mL and the second ligand concentration is 3.94×10¹⁰ to 6.31×10¹¹ molecules/mL.

In an embodiment, the method further comprises a step of enriching the CD4+CD8+ cells for CD8α or CD8β.

In an embodiment, the immobilized Notch signalling ligand is DL4.

In an embodiment, the antigen is a CD19 polypeptide and the antigen receptor binds the CD19 polypeptide.

In an embodiment, the CD19 polypeptide is an engineered variant of CD19.

In an embodiment, the antigen is a polypeptide having the sequence of SEQ ID NO:8.

In an embodiment, the substrate is a particle.

In an embodiment, the particle is comprised of a material selected from a group consisting of polystyrene, iron oxide and gold.

In an embodiment, the particle is comprised of polystyrene and magnetizable iron oxides.

In an embodiment, the antigen is covalently conjugated to the particle.

In an embodiment, the step of contacting the population of CD4+CD8+ cells expressing the antigen receptor with the antigen coupled to the particle is conducted at a particle:cell ratio of 1:1.

In a second aspect of the disclosure, a population of CD4−CD8+ T cells made according to the methods of the first aspect is provided.

In an embodiment, the population of CD4−CD8+ T cells express a chimeric antigen receptor (CAR).

In an embodiment, the CAR is a CD19 CAR, and wherein the antigen is a CD19 polypeptide.

In an embodiment, the population of CD4−CD8+ T cells is derived in vitro from pluripotent stem cells.

In an embodiment, the population of CD4−CD8+ T cells is TRAC−/− and/or CD3+.

In an embodiment, the population of CD4−CD8+ cells is greater than 50% CD8αα+.

In a third aspect, a method of inducing cytotoxicity of a tumor cell is provided. The method comprises exposing the tumor cell to a population of CD4−CD8+ T cells. The population of CD4−CD8+ T cells is made according to the method of the first aspect.

In a fourth aspect, a method of treating a disease or condition in a subject is provided. The method comprises generating CD4−CD8+ T cells according to the method of the first aspect and administering an effective amount of the CD4−CD8+ T cells to a subject in need thereof.

In a fifth aspect, a use of CD4−CD8+ T cells in the manufacture of a medicament for the treatment of a disease or condition is provided. The CD4−CD8+ T cells are generated by a method according to the first aspect.

In a sixth aspect, a cell stimulatory particle is provided. The cell stimulatory particle comprises a particle and a modified antigen immobilized on a surface of the particle. The modified antigen comprises one or more amino acid substitutions from a native antigen. The modified antigen has greater solubility and/or stability in vitro in comparison to the native antigen.

In an embodiment, the modified antigen comprises an extracellular domain of an antigen.

In an embodiment, the modified antigen is covalently conjugated to the particle.

In an embodiment, the particle is comprised of a material selected from a group consisting of polystyrene, iron oxide and gold.

In an embodiment, the particle is comprised of polystyrene and magnetizable iron oxide.

In an embodiment, the modified antigen is a CD19 antigen.

In an embodiment, the modified antigen is a polypeptide having the sequence of SEQ ID NO:8.

### BRIEF DESCRIPTION OF THE DRAWINGS

In order that the subject matter may be readily understood, embodiments are illustrated by way of non-limiting examples in the accompanying drawings.

FIG. 1A is an annotated image of a sodium dodecyl-sulfate polyacrylamide gel electrophoresis (SDS-PAGE) gel of CD19 protein variants under both non-reducing (−) and reducing (+) conditions.

FIG. 1B is a graph quantifying the binding of the CD19 antibody FMC63 on 2D-coated CD19-Fc and CD19.1-Fc, as measured by direct enzyme-linked immunosorbent assay (ELISA).

FIG. 1C is a graph quantifying the thermal melting curve of CD19 protein variants (top) and the first derivative plot of the melting curve (bottom).

FIG. 2 is a graph quantifying binding of FITC-labeled CD19.1 to primary anti-CD19 CAR-T cells. MFI, mean fluorescent intensity; conc., concentration.

FIG. 3 is a graph quantifying the density of CD19.1-Fc and CD19.1-His protein on beads, as measured by bicinchoninic assay (BCA).

FIG. 4 are graphs quantifying expression of CD69 in primary unmodified T cells (top) and primary CD19 CAR-T cells (bottom) with CD19-coated beads or alternate activation methods, as assessed by flow cytometry.

FIG. 5A are graphs quantifying cell expansion (top) and viability (bottom) in primary CD19 CAR-T cells with cytokines and CD19-coated beads (“IL-2 CD19”; “IL7/IL-15 CD19”) or alternate activation methods.

FIG. 5B are flow cytometry plots of marker expression for unstimulated (left) or CD19-coated bead-stimulated (right) primary CD19 CAR-T cells.

FIG. 5C is a graph quantifying fold expansion of primary CD19 CAR-T cells following 6 days of culture with cytokines and CD19-coated beads or alternate activation methods.

FIG. 6A is a graph quantifying CD19 CAR-Jurkat cell activation by soluble OKT3 or CD19.1-Fc.

FIG. 6B is a graph quantifying CD19 CAR-Jurkat cell activation by 2D-coated CD19.1-Fc in comparison to other stimulation conditions.

FIG. 6C is a graph quantifying CD19 CAR-Jurkat cell activation by 2D-coated CD19.1-His in comparison to other stimulation conditions.

FIG. 6D is a graph quantifying CD19 CAR-Jurkat cell activation by 2D-coated FMC63 in comparison to 2D-coated CD19.1-His and other stimulation conditions.

FIG. 7 is a graph quantifying luminescence response of 41BB-NFAT-Jurkat cells to stimulation with CD19.1-coated beads in comparison to other stimulation conditions.

FIG. 8 is a graph quantifying luminescence response of 41BB-NFAT-Jurkat cells to stimulation with CD19.1-coated beads in comparison to other stimulation conditions.

FIGS. 9A-9C are graphs quantifying the half-maximal effective concentration (EC50) of binding of FMC63 anti-CD19 antibody to: (FIG. 9A) CD19.1-Fc coated beads; (FIG. 9B) CD19.1-His coated beads; and (FIG. 9C) wild-type (wt) CD19-His coated beads.

FIG. 10A is a graph quantifying viability of iPSC-derived CAR+ T lineage cells following culture with commercial DL4 coating (StemSpan™ Lymphoid Differentiation Coating Material, STEMCELL Technologies) or immobilized Notch ligand engineered thymic niche (2D ETN) and CD19-coated beads at varying ratios.

FIG. 10B is a graph quantifying fold expansion of iPSC-derived CAR+ T lineage cells following culture with commercial DL4 coating or 2D ETN and CD19-coated beads at varying ratios.

FIG. 10C is a graph quantifying viability of iPSC-derived CAR+ CD4−CD8+ (CD8SP) cells following culture with commercial DL4 coating or 2D ETN and CD19-coated beads at varying ratios.

FIG. 10D is a graph quantifying the percentage of iPSC-derived CAR+ CD4−CD8+ (CD8SP) cells generated following culture with commercial DL4 coating or 2D ETN and CD19-coated beads at varying ratios.

FIG. 11A is a graph quantifying the percentage of iPSC-derived CAR+ cells that are CD8αα+(CD8αα, dark bars) and CD8αβ+ (CD8ab, light bars) generated following culture with commercial DL4 coating and CD19-coated beads at varying ratios.

FIG. 11B is a graph quantifying the percentage of iPSC-derived CAR+ cells that are CD8αα+(CD8αα, dark bars) and CD8αβ+ (CD8ab, light bars) generated following culture with 2D ETN and CD19-coated beads at varying ratios.

FIG. 12A is a graph quantifying the cytotoxicity of iPSC-derived T lineage cells generated via various methods, as assessed by a co-culture assay with CD19+ Raji cells (WT Raji) at varying effector to target (E:T) ratios.

FIG. 12B is a graph quantifying the cytotoxicity of iPSC-derived T lineage cells generated via various methods, as assessed by a co-culture assay with CD19+ Raji cells (WT Raji) at varying effector to target (E:T) ratios.

FIG. 13A is a graph quantifying the cytotoxicity of the bulk population of iPSC-derived CAR+ T lineage cells against CD19+ Raji cells (WT Raji, dark bars) or CD19+ Raji cells (WT Raji, light bars), in comparison with primary untransduced (Primary Untxd T) and primary CD19 CAR-transduced (Primary CAR T) cells.

FIG. 13B is a flow cytometry plot depicting marker expression and sorting strategy for iPSC-derived CAR+ T lineage cells. Outlines indicate CD4−CD8α− (double-negative, DN, bottom left quadrant) and CD4−CD8α+ (bottom right quadrant) cell populations.

FIG. 13C is a graph quantifying the cytotoxicity of sorted iPSC-derived DN (CD8−CD4− iPSC CAR 5 DN) and CD4−CD8+ (CD8+CD4− iPSC CAR SP) cell populations against CD19+ Raji cells (WT Raji, dark bars) or CD19+ Raji cells (WT Raji, light bars), in comparison with primary CD19 CAR-transduced (Primary CD8+CD4-CAR T) cells.

FIG. 14A are flow cytometry plots depicting marker expression of CAR-modified iPSC-derived T lineage cells cultured for 14 days on 2D ETN without CD19-coated beads.

FIG. 14B is a graph quantifying viability of CAR-modified iPSC-derived T lineage cells cultured for 14 days on 2D ETN without CD19-coated beads.

FIG. 14C is a graph quantifying CAR expression for CAR-modified iPSC-derived T lineage cells cultured for 14 days on 2D ETN without CD19-coated beads.

FIG. 15A are flow cytometry plots depicting marker expression for the CAR-modified iPSC-derived T lineage cells shown in FIG. 14 following extended culture in unstimulated (top) or CD19-coated bead-stimulated (bottom) conditions.

FIG. 15B is a graph quantifying viability of the CAR-modified iPSC-derived T lineage cells shown in FIG. 14 following extended culture in unstimulated or CD19-coated bead-stimulated conditions.

FIG. 16A is a graph quantifying viability of iPSC-derived T lineage cells cultured with CD19-coated beads.

FIG. 16B is a graph quantifying fold expansion over time of iPSC-derived T lineage cells cultured with CD19-coated beads.

FIG. 17 are flow cytometry plots depicting marker expression for iPSC-derived T lineage cells cultured with CD19-coated beads.

FIG. 18A are flow cytometry plots depicting marker expression for iPSC-derived T lineage cells cultured with CD19-coated beads.

FIG. 18B is a graph quantifying cytotoxicity, as assessed by WT and CD19 KO Raji target cell expression of luciferase, for iPSC-derived T lineage cells cultured with CD19-coated beads in comparison with primary T cell and target cell controls.

FIG. 19 are graphs quantifying cytotoxicity of iPSC-derived T lineage cells cultured with CD19-coated beads in comparison with primary T cell and target cell controls, as assessed by co-culture with A549 CD19+ cells (left) or A549 CD9− cells (right) at varying effector to target (E:T) ratios.

FIG. 20A is a graph quantifying cytotoxicity of iPSC-derived T lineage cells cultured with CD19-coated beads in comparison with primary T cell and target cell controls at varying effector to target (E:T) ratios. The first round of a serial restimulation assay with target cells (“Stim #1 Count”) is shown.

FIG. 20B is a graph quantifying viability of iPSC-derived T lineage cells cultured with CD19-coated beads in comparison with primary T cell and target cell controls at varying effector to target (E:T) ratios. The first round of a serial restimulation assay with target cells (“Stim #1 viability”) is shown.

FIG. 20C is a graph quantifying cytotoxicity of iPSC-derived T lineage cells cultured with CD19-coated beads in comparison with primary T cell and target cell controls at varying effector to target (E:T) ratios. The second round of a serial restimulation assay with target cells (“Stim #2 Count”) is shown.

FIG. 21 is graphs quantifying cytotoxicity of iPSC-derived T lineage cells cultured with CD19-coated beads in comparison with primary T cell and target cell controls at varying effector to target (E:T) ratios in a serial restimulation assay.

FIG. 22 is graphs quantifying the expression of 4, 3, 2, 1, or no (0) exhaustion markers (TIGIT, TIM3, LAG3, PD-1) of iPSC-derived T lineage cells cultured with CD19-coated beads in comparison with primary T cells following the first round of the serial restimulation assay shown in FIG. 21.

FIG. 23A is a graph quantifying viability of iPSC-derived T lineage cells after 24 hours of CD19 bead stimulation with 2D or 3D ETN (day 19).

FIG. 23B is a graph quantifying cell marker expression (CD4−CD8+, CD8 SP; CD4+CD8+, DP; CD4+CD8−, CD4 ISP; CD4−CD8−, DN) of iPSC-derived T lineage cells after 24 hours of CD19 bead stimulation with 2D or 3D ETN (day 19).

FIG. 23C is a graph quantifying percentage of CD8αα+(CD8 aa) or CD8αβ+ (CD8 ab) cells for iPSC-derived T lineage cells after 24 hours of CD19 bead stimulation with 2D or 3D ETN (day 19).

FIG. 23D is a graph quantifying viability of iPSC-derived T lineage cells after 48 hours of recovery following CD19 bead stimulation with 2D or 3D ETN (day 21).

FIG. 23E is a graph quantifying cell marker expression (CD4−CD8+, CD8 SP; CD4+CD8+, DP; CD4+CD8−, CD4 ISP; CD4−CD8−, DN) of iPSC-derived T lineage cells after 48 hours of recovery following CD19 bead stimulation with 2D or 3D ETN (day 21).

FIG. 23F is a graph quantifying percentage of CD8αα+(CD8 aa) or CD8αβ+(CD8 ab) cells for iPSC-derived T lineage cells after 48 hours of recovery following CD19 bead stimulation with 2D or 3D ETN (day 21).

FIG. 24A is a graph quantifying CAR expression for iPSC-derived T lineage cells after 24 hours of CD19 bead stimulation with 2D or 3D ETN.

FIG. 24B is a graph quantifying CAR expression for iPSC-derived T lineage cells after 48 hours of recovery following CD19 bead stimulation with 2D or 3D ETN (day 21).

FIG. 25 is a schematic of the stages of in vitro differentiation of CD8+ cells from iPSCs, including a summary of culture conditions at each stage.

FIG. 26A is a graph quantifying cell viability of cells through 28 days of differentiation from iPSC-derived HSPCs under a 2D (“NTX4B3-4 2D”, solid lines) or 3D (“NTX4B3-4 3D”, dashed lines) differentiation process.

FIG. 26B is a graph quantifying cumulative fold expansion of cells through 28 days of differentiation from iPSC-derived HSPCs under a 2D (“NTX4B3-4 2D”, solid lines) or 3D (“NTX4B3-4 3D”, dashed lines) differentiation process.

FIG. 27 is flow cytometry plots of marker expression of NTX4B3 cells cultured under 2D differentiation conditions for 17 or 24 days.

FIG. 28 is flow cytometry plots of marker expression of NTX4B3 cells cultured under 3D differentiation conditions for 17, 24, or 28 days.

FIG. 29A is a graph quantifying cell viability following culture of iPSC-derived DPs with antigen beads.

FIG. 29B is a graph quantifying cumulative fold expansion following culture of iPSC-derived DPs with antigen beads.

FIG. 30 is flow cytometry plots of marker expression of NTX4B3 cells cultured with antigen beads at day 35.

FIG. 31 is a graph quantifying results of an in vitro serial restimulation assay of primary CD8+ CAR-T cells and iPSC-derived CD8+ CAR-T cells generated with antigen-coated beads. Normalized green area (vertical axis) represents the number of CD19+/+ or CD19−/− target cells for each culture condition.

FIG. 32 is flow cytometry plots of marker expression of CD8+ cells generated from iPSCs generated with antigen-coated beads.

FIG. 33 is a graph quantifying cumulative cytotoxicity and fold expansion of primary CD8+ CAR-T cells and iPSC-derived CD8+ CAR-T cells generated with antigen-coated beads during an in vitro serial restimulation assay.

FIG. 34 is flow cytometry plots of marker expression of iPSC-derived CD8+ CAR-T cells generated with antigen-coated beads before (top) and after (bottom) an in vitro serial restimulation assay.

FIG. 35A is a graph quantifying memory cell marker expression for primary CD8+ CAR-T cells and iPSC-derived CD8+ CAR-T cells generated with antigen-coated beads before (“Baseline”) and after (“Stim 4”) an in vitro serial restimulation assay. Cells were assigned into various memory subsets based on the following expression profile: T stem cell memory (TSCM), CD62L+CD45RA+CD95+; T central memory (TCM), CD62L+CD45RA−CD45RO+; T effector memory (TEM), CD62L−CD45RA−CD45RO+; Terminally differentiated effector memory cells re-expressing CD45RA (TEMRA), CD62L−CD45RA+CD45RO+. The percentage of cells in each memory subset is indicated.

FIG. 35B is a graph quantifying exhaustion marker expression for primary CD8+ CAR-T cells and iPSC-derived CD8+ CAR-T cells generated with antigen-coated beads before (“Baseline”) and after (“Stim 4”) an in vitro serial restimulation assay. The percentage of cells expressing various exhaustion markers are indicated.

FIG. 36A is a graph quantifying secretion of the effector cytokine interferon-γ (IFN-γ) for primary and iPSC-derived CD8+ CAR-T cells generated with antigen-coated beads during an in vitro serial restimulation assay.

FIG. 36B is a graph quantifying secretion of the effector cytokine Granzyme B for primary and iPSC-derived CD8+ CAR-T cells generated with antigen-coated beads during an in vitro serial restimulation assay.

FIG. 36C is a graph quantifying secretion of the effector cytokine TNF-α for primary and iPSC-derived CD8+ CAR-T cells generated with antigen-coated beads during an in vitro serial restimulation assay.

FIG. 37 is a graph quantifying cumulative fold expansion for primary CD8+ CAR-T cells and iPSC-derived CD8+ CAR-T cells generated with antigen-coated beads during an in vitro serial restimulation assay.

### DETAILED DESCRIPTION OF THE DISCLOSURE

Unless defined otherwise, all technical and scientific terms used herein have the same meaning as commonly understood by one of ordinary skill in the art to which this disclosure belongs.

Generally, the present disclosure provides cell stimulatory particles, methods of generating CD4−CD8+ cell populations from stem/progenitor cells using cell stimulatory particles, CD4−CD8+ cell populations generated by the methods disclosed herein; and a use of a CD4−CD8+ cell population in the manufacture of a medicament for the treatment of a disease or condition.

### Definitions

As used herein, the term “stem cell” refers to a cell that can differentiate into more specialized cells and has the capacity for self-renewal. Stem cells include pluripotent stem cells (PSCs), such as embryonic stem cells (ESCs) and induced pluripotent stem cells (iPSCs), and multipotent stem cells, such as mobilized peripheral blood-derived CD34+ stem cells, umbilical cord blood stem cells, and adult stem cells, which are found in various tissues. Methods for obtaining, deriving or producing stem cells are known in the art.

As used herein, the term “progenitor cell” refers to a cell that can differentiate into one or more types of cells, but typically has a limited capacity for self-renewal. Progenitor cells are derivatives of stem cells and have more limited potency relative to their corresponding source stem cells. For example, hematopoietic stem cells (HSCs), found in adult bone marrow, peripheral blood (in smaller numbers) and in umbilical cord blood, have the capacity to give rise to all other blood cells. Hematopoietic progenitor cells are multipotent or lineage-committed cells derived from HSCs that have the capacity to give rise to a more limited or specific type of blood cell. Hematopoietic stem/progenitor cells (HSPCs) typically exist as a heterogeneous population in vivo and have use as a heterogeneous population as described herein. HPCs and HSPCs may be characterized by expression of one or more of CD34, CD43, CD31 and CD45.

As used herein, the terms “progenitor T cell” and “proT cell” refer to a cell that is derived from a pluripotent stem cell or a CD34+ hematopoietic stem and/or progenitor cell and expresses at least CD7+, and has the capacity to differentiate into one or more types of immature and mature T cells. Examples of progenitor T cells include, but are not limited to, CD7+ cells, CD7+CD5+ cells, CD7+CD5+CD34+ cells, CD7+CD5+CD45RA+ cells, and/or CD7+CD5+CD1a+ cells.

As used herein, an “immature T cell” or mature T cell is a T lineage cell derived from a progenitor T cell. T cell development may be characterized by the progressive expression of cell surface receptors, particularly CD4 and CD8. In vivo, T lineage cells progress from progenitor T cells through CD4−CD8− (double-negative, DN), CD4+CD8− (CD4 immature single-positive, CD41SP), early CD4+CD8+ (double-positive, DP), late DPs, and CD4−CD8+ (CD8 single-positive, CD8SP) and CD4 single-positive (CD4SP) stages. Late DPs are characterized by the presence of CD4+/CD8A+/CD8B+/CD3+ and TCRαβ+. The TCRγδ fate is not open at the late DP stage, the cell size decreases, and the cells are metabolically quiescent. In CD8 SPs, CD8 may be expressed as a heterodimer of CD8α and CD8β, resulting in CD8αβ+ cells, or as CD8αα homodimer, resulting in CD8αα+ cells. CD4−CD8+ cells may also be characterized by cell-surface expression of CD3 and one of TCRγδ (γδ T cells) or TCRαβ (αβ T cells).

As used herein, “serum-free medium” refers to a cell culture medium that lacks animal serum. Serum-free medium may include specific, known serum components isolated from an animal (including human animals), such as, for example, bovine serum albumin (BSA).

As used herein, “Notch signalling ligand” refers to any ligand capable of interacting with a Notch protein receptor for regulation of T cell lineage commitment and differentiation. Examples of Notch signalling ligands include Delta-like 4 (DL4), Delta-like-1 (DL1), Delta-like 3 (DL3), Jagged1 and Jagged2.

As used herein, Notch signalling ligand, for example, “Delta-like-4” and “DL4” refer to a protein that in humans is encoded by the DLL4 gene. DL4 is a member of the Notch signalling pathway and is also referred to in the art as “Delta like ligand 4” and “DLL4”. Herein, reference to DL4 is not limited to the entire DL4 protein, but includes at least the signaling peptide portion of DL4. For example, a commercially available product (Sino Biologicals) comprising the extracellular domain (Met 1-Pro 524) of human DL4 (full-length DL4 accession number NP 061947.1; SEQ ID NO: 1) fused to the Fc region of human IgG1 at the C-terminus is a DL4 protein suitable for use in the methods provided herein.

As used herein, Notch signalling ligand also includes a variant of a known Notch signalling ligand, for example, DL4. A variant Notch signalling ligand refers to a protein molecule which differs in amino acid sequence from the wild-type amino acid sequence by one or more additions, deletions, and/or substitutions and retains the desired Notch signalling activity of the wild type DL4. Also included within the definition are variants such as polypeptides, oligopeptides, peptides and proteins having amino acid sequence identity to a given polypeptide, oligopeptide, peptide or protein. The percent identity can be, for example, at least about 50%, 55%, 60%, 65%, 70%, 75%, 80%, 85%, 90% 91%, 92%, 93%, 94%, 95%, 96%, 97%, 98%, 99%, or 100% amino acid identity to the given polypeptide, oligopeptide, peptide or protein over a specified length, for example, over the full length of the polypeptide.

As used herein, “Vascular cell adhesion molecule 1” and “VCAM-1” refer to a protein that in humans is encoded by the VCAM1 gene. VCAM-1 is a cell surface sialoglycoprotein, a type I membrane protein that is a member of the Ig superfamily. VCAM-1 is also referred to in the art as “vascular cell adhesion protein 1 and cluster of differentiation 106” (CD106). Herein, reference to VCAM-1 is not limited to the entire VCAM-1 protein, but includes at least the signaling peptide portion of VCAM-1 (QIDSPL (SEQ ID NO: 2) or TQIDSPLN (SEQ ID NO: 3)). For example, a commercially available mouse VCAM-1-Fc chimeric protein (R&D) that comprises (Phe25-Glu698) region of mouse VCAM-1 (full-length murine VCAM-1 accession number CAA47989; SEQ ID NO: 4) fused with the Fe region of human IgG1 is a VCAM-1 protein suitable for use herein. Use of at least a portion of human VCAM-1 (full-length human VCAM-1 accession number P19320, NP001069, EAW72950; SEQ ID NO: 5) may also be suitable for use in the methods provided herein. Herein, reference to VCAM-1 also includes a variant, which differs in amino acid sequence from the wild-type amino acid sequence of VCAM-1 by one or more additions, deletions, and/or substitutions and retains the desired activity of the wild type VCAM-1. Also included within the definition are variants such as polypeptides, oligopeptides, peptides and proteins having amino acid sequence identity to a given polypeptide, oligopeptide, peptide or protein. The percent identity can be, for example, at least about 50%, 55%, 60%, 65%, 70%, 75%, 80%, 85%, 90% 91%, 92%, 93%, 94%, 95%, 96%, 97%, 98%, 99%, or 100% amino acid identity to the given polypeptide, oligopeptide, peptide or protein over a specified length, for example, over the full length of the polypeptide. VCAM-1 has been shown to synergistically increase Notch signalling in combination with DL4 (e.g., Shukla et al., 2017).

As used herein, “two-dimensional engineered thymic niche (2D ETN)” refers to a two-dimensional substrate immobilized with a Notch signalling ligand, for example, DL4, and optionally VCAM-1. A two-dimensional (2D) substrate can include, for example, a tissue culture plate. Methods of immobilizing Notch signalling ligands on a 2D substrate are known in the art and are described, for example in Shukla et al., 2017.

As used herein, “three-dimensional engineered thymic niche (3D ETN)” or “ETN bead” refers to a three-dimensional substrate immobilized with a Notch signalling ligand, for example, DL4 and optionally VCAM-1.

As used herein, “antigen-presenting particle” or “antigen-coated bead” or “antigen bead” refers to an antigen immobilized on a substrate for example, on a three-dimensional substrate such as a particle or bead. The antigen may be immobilized on a substrate through covalent or non-covalent interactions, affinity-based interactions, or other suitable forms of interactions. For example, the antigen may be a CD19 antigen (“CD19-coated bead”, “CD19 bead”).

A three-dimensional (3D) substrate can include, for example, micron-size particles (or beads), with or without a magnetic core, coated with one or more full proteins, protein domains (e.g., extracellular, intracellular, or other domain), peptides or protein fragments. Several approaches can be used individually or in combination to produce protein coated particles, e.g.: physisorption driven by protein affinity to the particle material, chemical conjugation by reaction with, among others, amine, carboxyl, thiol, epoxy, azide reactive groups, or by coating an appropriate ligand to capture the protein of interest by affinity. Examples of affinity tags include but are not limited to: polyhistidine (His), Fc, biotin, Halo, aldehyde, Snap, Spy-Catcher and VIPER. Particles or beads may be composed of, for example, polystyrene, iron oxide, polystyrene and magnetizable iron oxides (magnetic polystyrene), gold, or other suitable materials known in the art. ETN and antigen beads may be used for culturing cells on a tissue culture plate, flasks, or other vessels utilized for culturing cells.

As used herein, “immobilized” or “surface-bound” refers to a ligand, such as a Notch signalling ligand or integrin ligand, antigen, peptide, or protein binding to a substrate through covalent or non-covalent interactions, affinity-based interactions, or other suitable forms of interactions.

As used herein, “enriched” cell population refers to when the cell population comprising one or more cell phenotypes (for example, CD4−CD8+ (CD8SP), CD4+CD8+ (DP), CD4+CD8− (CD4 ISP), CD4−CD8− (DN)) exhibits a higher absolute number or ratio of one of the cell phenotypes (for example, CD4−CD8+ (CD8SP)) compared to other cell phenotypes, where at least 25% of the cell population is comprised of a single cell phenotype.

As used herein, the term “subject” refers to a vertebrate, preferably a mammal (e.g., a non-human mammal), more preferably a primate and still more preferably a human. Mammals include, but are not limited to, primates, humans, farm animals, sport animals, and pets.

As used herein, the term “treatment”, “treat” or “treating” is an approach for obtaining beneficial or desired clinical results. For purposes of this disclosure, beneficial or desired clinical results include, but are not limited to, one or more of the following: increased immune response, increased T cell response, decreased extent of damage from a disease, condition, or disorder, decreased duration of a disease, condition, or disorder, and/or reduction in the number, extent, or duration of symptoms related to a disease, condition, or disorder. The term includes the administration of the compounds, agents, drugs or pharmaceutical compositions of the present disclosure to prevent or delay the onset of one or more symptoms, complications, or biochemical indicia of a disease or condition; lessening or improving one or more symptoms; shortening or reduction in duration of a symptom; or arresting or inhibiting further development of a disease, condition, or disorder. Treatment may be prophylactic (to prevent or delay the onset of a disease, condition, or disorder, or to prevent the manifestation of clinical or subclinical symptoms thereof) or therapeutic suppression or alleviation of symptoms after the manifestation of the disease, condition, or disorder. The beneficial or desired clinical result may be an increase or decrease (as appropriate) of 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to an appropriate control, for example, a subject that did not receive the therapy.

The term “administering” or “administration” as used herein refers to the placement of an agent, a drug, a compound, or a pharmaceutical composition as disclosed herein into a subject by a method or route which results in at least partial delivery of the composition to a desired site. The compounds and pharmaceutical compositions disclosed herein can be administered by any appropriate route which results in an effective treatment in the subject. Routes of administration of the compounds and pharmaceutical compositions disclosed herein include, but are not limited to, intravenous, or intraperitoneal routes of administration, or a combination thereof.

The term “effective amount” or “therapeutically effective amount”, for example an effective amount or therapeutically effective amount of a T cell lineage population as used herein is an amount sufficient to bring about any one or more beneficial or desired results. In more specific aspects, an effective amount may alleviate or ameliorate one or more symptoms of a disease; decrease the duration of time that one or more symptoms of a disease, are present in a subject; increase the survival rate of a subject having a disease. For prophylactic use, beneficial or desired results may include eliminating or reducing the risk, lessening the severity, or delaying the onset of a disease, including biochemical and/or histological symptoms of the infection, its complications and intermediate pathological phenotypes presenting during development of the disease. For therapeutic use, beneficial or desired results may include clinical results such as reducing one or more symptoms of a disease; decreasing the dose or length of administration of other medications required to treat the disease; enhancing the effect and/or reducing the toxicity of another medication; delaying the progression of the disease in a subject, decreasing the duration of time that one or more symptoms of a disease, are present in a subject, and/or increasing the overall survival rate of a subject having a disease. An effective amount can be administered in one or more than one dose, round of administration, or course of treatment.

For purposes of this disclosure, an effective dosage of a cell population or a pharmaceutical composition is an amount sufficient to accomplish prophylactic or therapeutic treatment either directly or indirectly. As is understood in the clinical context, an effective dosage of a compound, or a pharmaceutical composition may or may not be achieved in conjunction with another agent, drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. The amount may vary from one subject to another and may depend upon one or more factors, such as, for example, subject gender, age, body weight, subject's health history, and/or the underlying cause of the disease, condition, or disorder to be prevented, inhibited and/or treated.

The term “pharmaceutically acceptable carrier, diluent, or excipient” as used herein includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 mL” is intended to encompass 1 mL, 2 mL, 3 mL, 4 mL, 5 mL, 1-2 mL, 1-3 mL, 1-4 mL, 1-5 mL, 2-3 mL, 2-4 mL, 2-5 mL, 3-4 mL, 3-5 mL, and 4-5 mL.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “consisting of” and its derivatives, as used herein, are intended to be closed terms that specify the presence of stated features, integers, steps, operations, elements, and/or components, and exclude the presence or addition of one or more other features, integers, steps, operations, elements and/or components.

### General Techniques

Unless otherwise defined herein, scientific and technical terms used in connection with the present disclosure shall have the meanings that are commonly understood by those of ordinary skill in the art. Generally, nomenclatures used in connection with, and techniques of, cell and tissue culture, molecular biology, immunology, microbiology, genetics and protein and nucleic acid chemistry and hybridization described herein are those well-known and commonly used in the art.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cales, eds., 1987); Current Protocols in Molecular Biology (F. M. Ausubel eta/., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3^{rd}. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999); and Immunobiology (C. A. Janeway and P. Travers, 1997).

### Hematopoietic Stem/Progenitor Cell Populations

Generally, the in vitro methods of generating CD4−CD8+ cell populations provided herein involve culturing hematopoietic stem and/or progenitor cells in the presence of Notch signalling ligands and antigen stimulation under conditions and for a time suitable for differentiation into T cell lineage populations.

Hematopoietic stem/progenitor cells (HSPCs) typically exist as a heterogeneous population in vivo and have use as a heterogeneous population as described herein. HPCs and HSPCs may be characterized by expression of one or more of CD34, CD43, CD31 and CD45.

In an embodiment, the HSPCs may be obtained from cord blood, peripheral blood or bone marrow or they may be derived in vitro from pluripotent stem cells such as embryonic stem cells (ESCs) or induced pluripotent stem cells (iPSCs) or other intermediate stem cells. In a preferred embodiment, the stem and/or progenitor cells are human cells. In an embodiment, the stem cells are mobilized peripheral blood-derived CD34+ cells. In a preferred embodiment, the HSPCs are derived in vitro from iPSCs. Methods of generating HSPCs from iPSCs are known in the art, for example, differentiation with appropriate medium conditions (e.g., Trotman-Grant et al., 2021).

Suitable techniques for analyzing cell surface markers are known to those of ordinary skill in the art, and may include, for example, flow cytometry as used herein, or immunocytochemistry. The number of cells and viability of the cells may be analyzed by techniques well known to those of ordinary skill in the art, and may include, for example use of an automated cell counter as disclosed herein.

### Cell Culture Systems

Cells may be cultured in types of cell culture systems known in the art, for example, cell culture plates, culture dishes, and bioreactors including stirred-tank reactors (STRs), rocking bag bioreactors, and other suitable cell culture formats. Cell culture may be carried out under static conditions, dynamic or agitated conditions, or a combination of static and dynamic conditions. Bioreactors can be any type of bioreactor known in the art and can use any type of processing/culturing conditions and methods, including, for example, batch processes, fed-batch processes, and perfusion culturing methods and conditions.

### Notch Ligand Substrates

In an embodiment, HSPCs are cultured in a two-dimensional culture system utilizing a suitable 2D substrate, which can include, for example, a standard culture plate coated with Notch signalling ligands, for example, DL4. The culture plate may also be coated with VCAM-1.

In an embodiment, cells are cultured in a three-dimensional culture system utilizing a suitable 3D substrate, which can include, for example, micron-size particles (or beads), with or without a magnetic core, coated with one or more full proteins, protein domains (e.g. extracellular, intracellular, or other domain), peptides or protein fragments to activate Notch signalling.

In one example, the Notch signalling ligand, for example, DL4, alone or in combination with VCAM-1, may be conjugated to polystyrene microbeads as described in Trotman-Grant et al., 2021, and WO2019157597.

In another example, 3D ETN beads may be manufactured by affinity capturing DL4 and VCAM-1 carrying the appropriate affinity tag on streptavidin or protein G coated beads, where beads are diluted to 0.1% solids in Dulbecco's phosphate-buffered saline (DPBS) without Ca2+ or Mg2+, supplemented with 0.05% BSA, and incubated with the protein solution (0.1×-20× protein molar excess) for 60 min at room temperature with continuous stirring. At the end of the incubation period, excess free protein is removed by magnetic separation followed by a buffer exchange. The procedure is repeated an additional four times, after which 3D ETN beads are concentrated ten-fold for storage.

Quantification of protein immobilization may be carried out according to methods known in the art such as, for example, the colorimetric bicinchoninic acid (BCA) assay, an immunofluorescence assay, or other known detection methods.

### T Cell Therapies

T cells have a broad range of therapeutic applications. T cells may be modified by, for example, conventional gene editing approaches such as nuclease editing or viral vector transduction, to express a chimeric antigen receptor (CAR), and/or an exogenous T cell receptor (TCR), to generate engineered T cell therapies (Weber et al., 2020). T cells derived from progenitor cells, including pluripotent stem cells, may be genetically engineered at the pluripotent or progenitor cell stage. Engineered T cell therapies have applicability in, for example, oncology and autoimmune disorders. In oncology, engineered T cell therapies have applicability in, for example, hematologic cancers, such as B cell lymphoma, B cell acute lymphoblastic leukemia and other B cell malignancies, multiple myeloma, and other hematologic cancers, as well as in solid tumours such as, for example, mesothelioma, adenocarcinomas, gliomas, and sarcomas (Weber et al., 2020). In autoimmune disorders, engineered T cell therapies have applicability in, for example, Type I diabetes, rheumatoid arthritis, multiple sclerosis, and other autoimmune disorders or conditions (Weber et al., 2020).

Engineered T cell therapies may target antigens known to be expressed on target cell types, including tumour cells or within tumour tissues. Chimeric antigen receptors (CARs) may be designed to target surface antigens or multivalent soluble antigens. The targeting ectodomain of the CAR may be a single-chain variable fragment (scFv), single-domain antibodies (single variable domain on a heavy chain, VHH), nanoantibodies, or other antigen-binding domain (Qu et al., 2022). CAR-T cell therapies may be directed towards multiple antigens using varying CAR designs or multiple CARs (Qu et al. 2022). Exemplary oncology antigens and corresponding cancer types for CAR-T cell therapies are listed in Table 1 below (Qu et al., 2022; Guha et al., 2022; Drougkas et al., 2023; Want et al., 2023).

TABLE 1

| Antigen/Antigen Type | Cancer Type |
|---|---|
| 5 T4 (TPBG) | ovarian cancer |
| AE1/2 | gastric cancer |
| AFP | hepatocellular carcinoma (HCC) |
| Alpha folate receptor | serous ovarian, Fallopian tube, or primary peritoneal cancer |
| | (epithelial cancer) |
| ALPPL2 (PLAP-like) | ovarian cancer |
| ANXA2 (ANX2L4, | ovarian cancer |
| CAL1H, LIP2, | |
| LPC2, LPC2D, P36) | |
| ARP2/3 (p41-ARC) | gastric cancer |
| AXL (UFO, ARK) | breast cancer, glioma, lung cancer |
| B7H3 (CD276) | colorectal cancer, esophageal cancer, Ewing's sarcoma, gastric |
| | cancer, glioma, hepatocellular carcinoma (HCC), lung cancer, |
| | melanoma, pancreatic cancer, prostate cancer, osteosarcoma, |
| | ovarian cancer |
| B7H6 | gastric cancer |
| BAFF-R (TNFRSF13C, | B-acute lymphoblastic leukemia (B-ALL) |
| CD268) | |
| BCMA (TNFRSF17, | chronic lymphocytic leukemia (CLL), multiple myeloma (MM) |
| CD269) | |
| BRCA | serous ovarian, Fallopian tube, or primary peritoneal cancer |
| | (epithelial cancer) |
| BT-001 | solid tumor |
| CA19-9 (sialyl-Lewis) | gastric cancer |
| CAIX | renal cell carcinoma (RCC) |
| CD117 (c-kit, KIT) | acute myeloid leukemia (AML), Ewing's sarcoma, osteosarcoma |
| CD123 (IL3RA) | acute myeloid leukemia (AML), B-acute lymphoblastic leukemia |
| | (B-ALL) |
| CD126 (IL6R, gp80) | melanoma, multiple myeloma (MM), prostate cancer |
| CD133 (Prominin-1, | breast cancer, colorectal cancer, Ewing's sarcoma, gastric cancer, |
| PROM1, AC133) | glioma, hepatocellular carcinoma (HCC), lung cancer, |
| | osteosarcoma, ovarian cancer, pancreatic cancer |
| CD138 (Syndecan, | multiple myeloma (MM) |
| SDC) | |
| CD147 (BSG) | glioma, hepatocellular carcinoma (HCC), lung cancer |
| CD16 (FCG3, FCGR3, | melanoma |
| IGFR3, IMD20) | |
| CD166 (ALCAM) | Ewing's sarcoma, osteosarcoma |
| CD171 | neuroblastoma |
| CD177 (NB1 GP, | Ewing's sarcoma, osteosarcoma |
| PRV-1) | |
| CD19 (B lymphocyte | B-acute lymphoblastic leukemia (B-ALL), B-cell non-Hodgkin's |
| surface antigen B4, | lymphoma (B-NHL), Hodgkin's lymphoma (HL), chronic |
| T cell surface antigen | lymphocytic leukemia (CLL), multiple myeloma (MM) |
| LEU-12) | |
| CD20 (B Lymphocyte | B-acute lymphoblastic leukemia (B-ALL), B-cell non-Hodgkin's |
| surface antigens B1, | lymphoma (B-NHL), chronic lymphocytic leukemia (CLL), Mantle |
| Bp35, leU-16, MS4A1) | cell lymphoma (MCL), melanoma |
| CD20/CD22 | Relapsed or refractory lymphoid cancers |
| CD22 (Siglec-2, | B-acute lymphoblastic leukemia (B-ALL), B-cell non-Hodgkin's |
| BL-CAM, T cell surface | lymphoma (B-NHL), T-cell non-Hodgkin's lymphoma (T-NHL) |
| antigen LEU-14) | |
| CD23 (Low affinity | chronic lymphocytic leukemia (CLL) |
| immunoglobulin, εFc | |
| receptor, BLAST-2, | |
| FcεRII, FCER2, | |
| CLEC4J) | |
| CD30 (TNF receptor | Hodgkin's lymphoma (HL), T-cell non-Hodgkin's lymphoma (T- |
| superfamily member 8, | NHL) |
| TNFRSF8, Ki-1 antigen) | |
| CD318 (CDCP 1) | pancreatic cancer |
| CD32b (FcγRII, | chronic lymphocytic leukemia (CLL) |
| FCGR2A, FCGR2B) | |
| CD33 (Siglec-3) | acute myeloid leukemia (AML), myeloid malignancies |
| CD38 (ADP | acute myeloid leukemia (AML), B-acute lymphoblastic leukemia |
| ribocyclase 1) | (B-ALL), multiple myeloma (MM) |
| CD4 (OKT4) | T-cell non-Hodgkin's lymphoma (T-NHL) |
| CD44 (Hermes, Pgp1, | hepatocellular carcinoma (HCC), ovarian cancer |
| H-CAM, Hutch) | |
| CD44v6 (Epican, | acute myeloid leukemia (AML), breast cancer, gastric cancer, |
| HUTCH-I, LHR, | head and neck squamous cell carcinoma (HNSCC), lung cancer, |
| ECMR-III) | multiple myeloma (MM) |
| CD47 (MER6, IAP) | lung cancer, ovarian cancer |
| CD5 (LEU1) | T-acute lymphoblastic leukemia (T-ALL), T-cell non-Hodgkin's |
| | lymphoma (T-NHL) |
| CD56 (NCAM1) | multiple myeloma (MM) |
| CD7 (LEU 9, GP40, | acute myeloid leukemia (AML), T-acute lymphoblastic leukemia |
| TP41) | (T-ALL), T-cell non-Hodgkin's lymphoma (T-NHL) |
| CD70 (CD27L) | acute myeloid leukemia (AML), breast cancer, esophageal cancer, |
| | head and neck squamous cell carcinoma (HNSCC), melanoma, |
| | ovarian cancer, pancreatic cancer, renal cell carcinoma (RCC) |
| CD72 | B-acute lymphoblastic leukemia (B-ALL) |
| CDH17 | colorectal cancer, gastric cancer |
| CDH6 | ovarian cancer |
| CEA (CEACAM5) | breast cancer, colorectal cancer, metastatic colorectal cancer, |
| | gastric cancer, hepatocellular carcinoma (HCC), lung cancer, |
| | pancreatic cancer/pancreatic-biliary tract cancer, peritoneal |
| | cancer, prostate cancer |
| CLDN18.2 (CLDN18, | esophageal cancer, gastric cancer, lung cancer, |
| Claudin 18, SFTA5) | pancreatic/pancreatic-biliary tract cancer |
| CLDN6 (Skullin) | ovarian cancer, testicular cancer |
| CLEC14A (EGFR5) | lung cancer |
| CLL1 (CLEC12A) | acute myeloid leukemia (AML) |
| CLTX | glioma |
| c-MET (HGFR) | breast cancer, colorectal cancer, gastric cancer, glioma, |
| | hepatocellular carcinoma (HCC), lung cancer |
| CSPG4 (NG2, MCSP, | breast cancer, glioma, head and neck squamous cell carcinoma |
| MCSPG, MSK16, | (HNSCC), melanoma |
| HMW-MAA, MEL-CSPG) | |
| CXCR1 | ovarian cancer |
| CXCR4 (CD184, Fusin, | lung cancer |
| NPYR, HSY3RR, LAP3, | |
| LCR1) | |
| CXCR5 (CD185) | B-cell non-Hodgkin's lymphoma (B-NHL) |
| DLL3 (SCDO1) | lung cancer |
| DR5 | hepatocellular carcinoma (HCC) |
| DSC2 | gastric cancer |
| EGFR and CD133 | pancreatic cancer/pancreatic-biliary tract cancer |
| EGFR(ERBB1) | breast cancer, colorectal cancer, Ewing's sarcoma, gastric cancer, |
| | head and neck squamous cell carcinoma (HNSCC), lung cancer |
| | (malignant pleural mesothelioma), osteosarcoma, pancreatic |
| | cancer/pancreatic-biliary tract cancer |
| EGFR806 | central nervous system tumor, pediatric glioma |
| EGFRIII | glioblastoma, gliosarcoma and brain tumor |
| EGFRvIII | glioblastoma, glioma, hepatocellular carcinoma (HCC), pancreatic |
| | cancer |
| EpCAM (CD326, | breast cancer, colorectal cancer, gastric cancer, hepatocellular |
| TACSTD1) | carcinoma (HCC), pancreatic cancer, prostate cancer |
| EphA10 | breast cancer |
| EphA2 (ECK, CTPA, | Ewing's sarcoma, glioma, lung cancer, osteosarcoma |
| ARCC2, CTPP1, | |
| CTRCT6) | |
| FAP (SIMP, DPPIV, | glioma, lung cancer (malignant pleural mesothelioma), pancreatic |
| FAPA) | cancer |
| FcμR | chronic lymphocytic leukemia (CLL) |
| FLT3 (CD135, FLK2) | acute myeloid leukemia (AML) |
| Fn14 | glioma |
| FR-a | ovarian cancer |
| FRα (FOLR1) | breast cancer, gastric cancer, lung cancer, ovarian cancer |
| FRβ | acute myeloid leukemia (AML), lung cancer |
| GD2 | breast cancer, Ewing's sarcoma, glioma, lung cancer, melanoma, |
| | osteosarcoma |
| GD3 | melanoma |
| GFRα4 (GDNF family | thyroid cancer |
| receptor alpha-4) | |
| Glypican-3 | liver cancer |
| gp100 | breast cancer, hepatocellular carcinoma (HCC), melanoma |
| GPC3 (DGSX, OCI5, | hepatocellular carcinoma (HCC), lung cancer, renal cell carcinoma |
| SDYS, SGB, SGBS1, MXR7) | (RCC) |
| GPRC5D | multiple myeloma (MM) |
| GUCY2C (GUC2C, STAR) | colorectal cancer, pancreatic cancer |
| HER2 (ERBB2) | breast cancer, colorectal cancer, esophageal cancer, Ewing's |
| | sarcoma, gastric cancer, glioblastoma, glioma/pediatric glioma, |
| | head and neck squamous cell carcinoma (HNSCC), hepatocellular |
| | carcinoma (HCC), melanoma, lung cancer, osteosarcoma, ovarian |
| | cancer, pancreatic cancer/pancreatic-biliary tract cancer |
| HLA-A2 | ependymoma |
| ICAM-1 (CD54) | breast cancer, , gastric cancer, thyroid cancer |
| IGF1R (CD221, JTK13, MGC18216) | Ewing's sarcoma osteosarcoma |
| IL13Rα2 (CD213A2, IL13BP, CT19) | glioblastoma, glioma, melanoma |
| IM83 | liver cancer |
| KK-LC-1 | serous ovarian, Fallopian tube, or primary peritoneal cancer |
| | (epithelial cancer) |
| L1-CAM (CD171, HSAS, MASA) | ovarian cancer |
| LewisY | acute myeloid leukemia (AML), lung cancer, multiple myeloma |
| | (MM) |
| LILRB4 (CD85, LILRB3, ILT5, LIR3) | acute myeloid leukemia (AML) |
| LMP1 | hematological malignancies |
| Mesothelin (MSLN) | acute myeloid leukemia (AML), breast cancer, cervical cancer, |
| | colorectal cancer, gastric cancer, lung cancer (malignant pleural |
| | mesothelioma), ovarian cancer, pancreatic cancer |
| MG7 | hepatocellular carcinoma (HCC) |
| MPL (MPF, SMRP) | acute myeloid leukemia (AML) |
| MUC1 (PEM, PUM, DF3, MAM-6) | breast cancer, cervical cancer, colorectal cancer, esophageal |
| | cancer, Ewing's sarcoma, gastric cancer, glioma, head and neck |
| | squamous cell carcinoma (HNSCC), hepatocellular carcinoma |
| | (HCC), lung cancer, osteosarcoma, ovarian cancer, pancreatic |
| | cancer, renal cell carcinoma (RCC) |
| MUC16 (CA125) | ovarian cancer |
| NKG2DL | acute myeloid leukemia (AML), breast cancer, colorectal cancer, |
| | Ewing's sarcoma, gastric cancer, glioma, hepatocellular |
| | carcinoma (HCC), multiple myeloma (MM), osteosarcoma, |
| | prostate cancer, T-acute lymphoblastic leukemia (T-ALL) |
| NRP-1 (VEGF165R, NRP) | gastric cancer |
| NY-ESO-1 (CTAG1B, ESO1, LAGE2) | esophageal cancer, lung cancer, melanoma, multiple myeloma |
| | (MM) |
| OR2H1 | lung cancer, ovarian cancer |
| P32 | glioma |
| PAK4 | glioma |
| PD-L1 (CD274, B7-H1) | breast cancer, gastric cancer, head and neck squamous cell |
| | carcinoma (HNSCC), hepatocellular carcinoma (HCC), lung cancer, |
| | melanoma |
| PDPN (GP36) | glioma |
| PODXL (PCLP, PCLP1, Gp200) | pancreatic cancer |
| PR1 | acute myeloid leukemia (AML) |
| PSCA | colorectal cancer, gastric cancer, lung cancer, pancreatic cancer/ |
| | pancreatic-biliary tract cancer, metastatic pancreatic cancer, |
| | prostate cancer, metastatic castration-resistant prostate cancer |
| PSMA, prostate-specific | prostate cancer, metastatic castration-resistant prostate cancer, |
| membrane antigen (GIG27, FOLH, | salivary gland cancers |
| NAALAD1, PSM) | |
| PTK7 (CCK4) | breast cancer, lung cancer, ovarian cancer |
| ROBO1 (DUTT1) | pancreatic cancer |
| ROR1 (NTRKR1) | breast cancer, chronic lymphocytic leukemia (CLL), Ewing's |
| | sarcoma, lung cancer, osteosarcoma |
| ROR2 | gastric cancer, pancreatic cancer, renal cell carcinoma (RCC) |
| Siglec-6 (CD327, OB-BP1) | acute myeloid leukemia (AML), chronic lymphocytic leukemia |
| | (CLL) |
| SLAMF3 (CD229, Ly9) | multiple myeloma (MM) |
| SLAMF7 (CS1, CD319, CRACC) | multiple myeloma (MM) |
| TAA-T | acute myeloid leukemia (AML), Hodgkin's lymphoma (HL), |
| | myelodysplastic syndrome (MDS), |
| TAG 72 (CA72-4) | colorectal cancer, metastatic colorectal cancer, gastric cancer, |
| | ovarian cancer |
| TEM8 (ATR, GAPO) | breast cancer |
| TGFβ (IBDIMDE) | hepatocellular carcinoma (HCC), lung cancer |
| Tim3 (CD366, HAVCR2) | acute myeloid leukemia (AML) |
| TRBC | T-cell non-Hodgkin's lymphoma (T-NHL) |
| TROP2 (TACSTD2, GA733-1, | breast cancer, gastric cancer, pancreatic cancer |
| M1S1) | |
| TRP1/2 | melanoma |
| TSHR (LGR3, CHNG1) | thyroid cancer |
| TSLPR (CRL2, CRLF2) | B-acute lymphoblastic leukemia (B-ALL) |
| TSPAN8 | pancreatic cancer |
| U87 | pancreatic cancer |
| uPAR (CD87) | gastric cancer, ovarian cancer |
| VEGFR-2 (FLK-1) | melanoma, renal cell carcinoma (RCC) |
| WT1 (WIT-1) | acute myeloid leukemia (AML) |
| ανβ3 (CD51, CD61, GP3A) | melanoma |
| κ light chain | B-cell non-Hodgkin's lymphoma (B-NHL), chronic lymphocytic |
| | leukemia (CLL), multiple myeloma (MM) |
| λ light chain | B-cell non-Hodgkin's lymphoma (B-NHL), chronic lymphocytic |
| | leukemia (CLL) |

TCR-T cell therapies target antigens expressed as peptide-human leukocyte antigen (HLA) complexes on the surface of a target cell. These targets may include tumour-associated antigens (TAAs) and tumour-specific antigens (TSAs) (Baulu et al., 2023). Exemplary oncology antigens and corresponding cancer types for TCR-T cell therapies are listed in Table 2 below (Baulu et al., 2023; Sun et al., 2021; Want et al., 2023).

TABLE 2

| Antigen/Antigen Type | Cancer Type |
|---|---|
| AFP | hepatocellular carcinoma (HCC) |
| CEA | colorectal cancer |
| CTAG1A (NY-ESO-1) | bladder carcinoma, breast cancer, esophageal cancer, gastric cancer, |
| | hepatocellular carcinoma (HCC), lung cancer, myeloma, melanoma, |
| | neoplasms, serous ovarian, Fallopian tube, or primary peritoneal |
| | cancer (epithelial cancer), synovial sarcoma |
| EBV | acute myeloid leukemia (AML), head and neck squamous cell |
| | carcinoma (HNSCC), nasopharyngeal carcinoma, neuroblastoma |
| gp100 | melanoma |
| HA-1H | juvenile myelomonocytic leukemia (JMML), recurrent acute |
| | biphenotypic leukemia, recurrent acute undifferentiated leukemia |
| HBV | HBV-hepatocellular carcinoma (HCC) |
| HERV-E | kidney cancer |
| HPV16-E6 | human papillomavirus (HPV)-16+ cancers (cervical, vulvar, vaginal, |
| | penile, anal, and oropharyngeal cancers) |
| HPV16-E7 | human papillomavirus (HPV)-16+ cancers (cervical, vulvar, vaginal, |
| | penile, anal, and oropharyngeal cancers), neoplasia |
| Kita-kyushu Lung Cancer | breast cancer, cervical cancer, gastric cancer, lung cancer |
| Antigen 1/cancer testis | |
| antigen (KK-LC-1) | |
| KRAS mutations e.g. KRAS | gastric cancer, gastrointestinal cancer, pancreatic cancer, pancreatic |
| G12D | ductal adenocarcinoma, |
| LMP2 | nasopharyngeal carcinoma, recurrent and metastatic |
| | nasopharyngeal carcinoma |
| MAGE family | bladder cancer, breast cancer, cervical cancer, head and neck |
| | squamous cell carcinoma (HNSCC), melanoma, metastatic non-small |
| | cell lung carcinoma (NSCLC), metastatic malignant solid neoplasm, |
| | renal cell carcinoma (RCC), uveal melanoma |
| MAGEA10 | non-small cell lung carcinoma (NSCLC) |
| MAGEA3 | esophageal cancer, melanoma, metastatic solid tumors, myeloma, |
| | synovial sarcoma |
| MAGEA4 | esophageal cancer, solid tumors |
| MAGEC2 | head and neck squamous cell carcinoma (HNSCC), melanoma, uveal |
| | melanoma |
| MART-1 (MLANA) | melanoma |
| MCPyV | Merkel cell carcinoma |
| Mesothelin (MSLN) | Metastatic pancreatic ductal adenocarcinoma, ovarian cancer |
| NY-ESO-1 | liposarcoma, melanoma, myeloma, malignant Peripheral Nerve |
| | Sheath Tumor (MPNST), osteosarcoma, serous ovarian, Fallopian |
| | tube, or primary peritoneal cancer (epithelial cancer), soft tissue |
| | sarcoma, synovial sarcoma |
| PMEL (gp100) | skin cancer, melanoma |
| PRAME | acute myeloid leukemia (AML), myelodysplastic syndrome (MDS), |
| | uveal melanoma |
| TAC-1 | HER2-positive solid tumors |
| TP53 | breast cancer, metastatic breast cancer, kidney cancer, melanoma |
| TSA | malignant epithelial neoplasms |
| WT1 | acute myeloid leukemia (AML), chronic myeloid leukemia (CML), |
| | myelodysplastic syndromes (MDS), pleural malignant, recurrent non- |
| | small cell lung carcinoma (NSCLC) |

T cell therapies, including stem cell-derived T cell therapies, may be genetically modified, for example, to eliminate endogenous TCR expression by knock-out of the T cell receptor a constant (TRAC) gene (TRAC−/−).

It is contemplated that the cell populations derived from culturing stem/progenitor cells with the method provided herein may be contained in pharmaceutical compositions.

It is further contemplated that the cell populations derived from culturing stem/progenitor cells using the method provided herein may be used to treat a disease or condition in a subject. By “treat” we mean administering to the subject an effective amount of cells, as provided herein, under conditions suitable for increasing the number of T cells in the subject, which may result in prevention, inhibition and/or therapeutic treatment of a medical condition. By “effective amount” we mean a therapeutically effective amount such as, for example, the amount of cells that, upon administration to a subject, is sufficient to achieve the intended purpose (e.g., treatment). The amount may vary from one subject to another and may depend upon one or more factors, such as, for example, subject gender, age, body weight, subject's health history, and/or the underlying cause of the condition to be prevented, inhibited and/or treated.

For example, subjects afflicted with an oncological or autoimmune disease, condition or disorder may benefit from administration of CD4−CD8+ cell populations, as described herein.

The pharmaceutical composition provided herein may be administered to a subject to treat cancer or autoimmune disorders in the subject.

The pharmaceutical composition provided herein may be administered to a subject in an effective amount or a therapeutically effective amount. A person of ordinary skill in the art would be able to determine such amounts based on such factors as the subject's size (e.g., weight), age and/or sex; the severity of the subject's symptoms; and the particular composition or route of administration selected. A person skilled the art would also know how to select the proper route of administration and to administer the compounds and compositions provided herein.

The dosage of the pharmaceutical composition of the disclosure varies depending on many factors, such as the pharmacodynamic properties of the composition, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, the pharmaceutical composition is administered initially in a suitable dosage that is adjusted as required, depending on the clinical response.

### Kits

The invention also provides kits comprising the pharmaceutical composition described herein. Kits of the invention include one or more containers comprising the pharmaceutical composition described herein and instructions for use in accordance with any of the methods of the invention described herein. Generally, these instructions comprise a description of administration of the pharmaceutical composition for the above-described therapeutic treatments. In some embodiments, kits are provided for producing a single-dose administration unit.

The instructions relating to the use of the pharmaceutical composition generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

### Antigen Bead Compositions

The base bead can be composed of any material suitable for covalent or non-covalent coating with functional proteins including polystyrene (PS particles) and styrene co-polymers such as maleic acid (SMA particles), acrylic acid (SAA particles), divinylbenzene (PS-DVB particles), acrylonitrile (PAN particles), butadiene (PBR particles) and vinyl acetate (PVA particles), polymethylmethacrylate (PMMA particles), silica (silica particles) including silica crosslinked with tetraethyl orthosilicate (TEOS) and/or doped with metal ions, and particles made from gold (gold particles).

### Bead Size

Beads may be of a size suitable for convenient handling in aqueous suspensions.

### Protein Coating

In addition to the methods described above, an antigen may be conjugated directly onto a carboxyl functionality on beads. Further, it is also contemplated that heterobifunctional PEG of varying lengths may be conjugated onto beads and then conjugated onto the end carboxyl group of the PEG. This approach may improve the shelf-life of CD19 beads and enable control of the potency of CD19 beads.

### Antigens

In addition to the antigens listed in Tables 1 and 2 above, it is contemplated that other antigens known in the art may be utilized.

It is also contemplated that the antigen-presenting particles described herein may include the signalling portion of an antigen such as, for example, the extracellular domain or the ectodomain of an antigen. The antigen may be an engineered or variant sequence from a native antigen. It is also contemplated that the antigen may be an antigen-mimetic peptide.

### Example 1: Generation of ETN and CD19.1-Coated Beads

### DL4 and VCAM-1 Production

Recombinant DL4-Fc fusion protein was purchased from Sino Biological or manufactured in-house using HEK-293T cells and purified with HiTrap™ Protein G affinity columns (GE Healthcare) as previously described (e.g., Trotman-Grant et al., 2017). Recombinant VCAM-1-FC fusion protein was purchased from R&D Systems. DL4 and VCAM-1 that are suitable for preparation of 2D ETN and 3D ETN as further described below, are shown in Table 3.

TABLE 3

| Protein | Species | Sequence |
|---|---|---|
| DL4 | Human | MAAASRSASGWALLLLVALWQQRAAGSGVFQLQLQEFINERGVLASGRPCEPGCRTFFRV |
| | | CLKHFQAVVSPGPCTFGTVSTPVLGTNSFAVRDDSSGGGRNPLQLPFNFTWPGTFSLIIEA |
| | | WHAPGDDLRPEALPPDALISKIAIQGSLAVGQNWLLDEQTSTLTRLRYSYRVICSDNYYGDN |
| | | CSRLCKKRNDHFGHYVCQPDGNLSCLPGWTGEYCQQPICLSGCHEQNGYCSKPAECLCRP |
| | | GWQGRLCNECIPHNGCRHGTCSTPWQCTCDEGWGGLFCDQDLNYCTHHSPCKNGATCS |
| | | NSGQRSYTCTCRPGYTGVDCELELSECDSNPCRNGGSCKDQEDGYHCLCPPGYYGLHCEH |
| | | STLSCADSPCFNGGSCRERNQGANYACECPPNFTGSNCEKKVDRCTSNPCANGGQCLNR |
| | | GPSRMCRCRPGFTGTYCELHVSDCARNPCAHGGTCHDLENGLMCTCPAGFSGRRCEVRT |
| | | SIDACASSPCFNRATCYTDLSTDTFVCNCPYGFVGSRCEFPVGLPPSFPWVAVSLGVGLAVL |
| | | LVLLGMVAVAVRQLRLRRPDDGSREAMNNLSDFQKDNLIPAAQLKNTNQKKELEVDCGL |
| | | DKSNCGKQQNHTLDYNLAPGPLGRGTMPGKFPHSDKSLGEKAPLRLHSEKPECRISAICSP |
| | | RDSMYQSVCLISEERNECVIATEV (SEQ ID NO: 1) |
| VCAM-1 | Human | QIDSPL (SEQ ID NO: 2) |
| VCAM-1 | Human | TQIDSPLN (SEQ ID NO: 3) |
| VCAM-1 | Mouse | MPVKMVAVLGASTVLWILFAVSQAFKIEISPEYKTIAQIGDSMALTCSTTGCESPLFSWRTQI |
| | | DSPLNAKVRTEGSKSVLTMEPVSFENEHSYLCTATCGSGKLERSIHVDIYSFPKDPEIQFSGPL |
| | | EVGKPVTVKCLAPDIYPVYRLEIDLFKGDQLMNRQEFSSEEMTKSLETKSLEVTFTPVIEDIGK |
| | | ALVCRAKLHIDQIDSTLKERETVKELQVYISPRNTTISVHPSTRLQEGGAVTMTCSSEGLPAP |
| | | EIFWGRKLDNEVLQLLSGNATLTLIAMRMEDSGVYVCEGVNLIGRDKAEVELVVQEKPFIV |
| | | DISPGSQVAAQVGDSVVLTCAAIGCDSPSFSWRTQTDSPLNGVVRNEGAKSTLVLSSVGFE |
| | | DEHSYLCAVTCLQRTLEKRTQVEVYSFPEDPVIKMSGPLVHGRPVTVNCTVPNVYPFDHLEI |
| | | ELLKGETTLMKKYFLEEMGIKSLETKILETTFIPTIEDTGKSLVCLARLHSGEMESEPKQRQSV |
| | | QPLYVNVAPKETTIWVSPSPILEEGSPVNLTCSSDGIPAPKILWSRQLNNGELQPLSENTTLT |
| | | FMSTKRDDSGIYVCEGINEAGISRKSVELIIQVSPKDIQLTVFPSKSVKEGDTVIISCTCGNVPE |
| | | TWIILKKKAKTGDMVLKSVDGSYTIRQAQLQDAGIYECESKTEVGSQLRSLTLDVKGKEHNK |
| | | DYFSPELLALYCASSLVIPAIGMIVYFARKANMKGSYSLVEAQKSKV (SEQ ID NO: 4) |
| VCAM-1 | Human | MPGKMVVILGASNILWIMFAASQAFKIETTPESRYLAQIGDSVSLTCSTTGCESPFFSWRTQ |
| | | IDSPLNGKVTNEGTTSTLTMNPVSFGNEHSYLCTATCESRKLEKGIQVEIYSFPKDPEIHLSGP |
| | | LEAGKPITVKCSVADVYPFDRLEIDLLKGDHLMKSQEFLEDADRKSLETKSLEVTFTPVIEDIG |
| | | KVLVCRAKLHIDEMDSVPTVRQAVKELQVYISPKNTVISVNPSTKLQEGGSVTMTCSSEGLP |
| | | APEIFWSKKLDNGNLQHLSGNATLTLIAMRMEDSGIYVCEGVNLIGKNRKEVELIVQEKPFT |
| | | VEISPGPRIAAQIGDSVMLTCSVMGCESPSFSWRTQIDSPLSGKVRSEGTNSTLTLSPVSFEN |
| | | EHSYLCTVTCGHKKLEKGIQVELYSFPRDPEIEMSGGLVNGSSVTVSCKVPSVYPLDRLEIELL |
| | | KGETILENIEFLEDTDMKSLENKSLEMTFIPTIEDTGKALVCQAKLHIDDMEFEPKQRQSTQT |
| | | LYVNVAPRDTTVLVSPSSILEEGSSVNMTCLSQGFPAPKILWSRQLPNGELQPLSENATLTLI |
| | | STKMEDSGVYLCEGINQAGRSRKEVELIIQVTPKDIKLTAFPSESVKEGDTVIISCTCGNVPET |
| | | WIILKKKAETGDTVLKSIDGAYTIRKAQLKDAGVYECESKNKVGSQLRSLTLDVQGRENNKD |
| | | YFSPELLVLYFASSLIIPAIGMIIYFARKANMKGSYSLVEAQKSKV (SEQ ID NO: 5) |

### Preparation of 2D ETN

Tissue culture plates with 6-well, 12-well, 24-well, 48-well or 96-well were coated with the Notch signalling ligand, DL4, and VCAM-1 overnight at 4° C. or for 3 hours at 37° C. Tissue culture plates may be stored at 4° C. for up to 2 weeks after coating. To coat, a solution of 20 μg/mL DL4 and 10 μg/mL VCAM-1 was prepared in Dulbecco's phosphate-buffered saline (DPBS) (−/−). The appropriate coating volume per well of DL4 and VCAM-1 diluted in DPBS were added to the tissue culture plates as shown in Table 4.

TABLE 4

| Plate format | Surface area (cm²/well) | Coating volume (L/well) |
|---|---|---|
| 96-well | 0.32 | 50 |
| 48-well | 0.95 | 150 |
| 24-well | 1.9 | 300 |
| 12-well | 4 | 650 |
| 6-well | 9.5 | 1500 |

The tissue culture plate(s) were tapped gently to ensure that the coating solution comprising the Notch signalling ligand, DL4, and VCAM-1 is evenly spread out throughout the well surface. The tissue culture plates were sealed with Parafilm® prior to being stored at 4° C. overnight or at 37° C. for 3 hours. Tissue culture plates coated with Notch signalling ligand DL4, and VCAM-1 overnight at 4° C. were placed in a 37° C. incubator for equilibration for three hours before plating cells. Following equilibration, or coating the tissue culture plates with the Notch signalling ligand DL4, and VCAM-1 for 3 hours at 37° C., the coating solution was aspirated from the wells. The wells were rinsed with DPBS (−/−) using the volumes shown in Table 5, immediately followed by addition of the cell suspension into the tissue culture plates.

TABLE 5

| | DPBS (-/-) wash volume |
|---|---|
| Plate format | (μL/well) |
| 96-well | 200 |
| 48-well | 800 |
| 24-well | 1600 |
| 12-well | 3200 |
| 6-well | 7500 |

### Preparation of 3D ETN

The dose of 3D ETN may be calculated to scale with the bead diameter, and may be expressed as: a dose proportional to the surface area of the culturing surface of the culture plate or flask (e.g., as in Table 6), the number of beads per unit volume of culture, or the bead surface area per unit volume of culture. As calculated on a per unit volume basis, the bead per mL concentration does not change for different vessels. A “1×” bead dose denotes complete coverage of the plate surface by one layer of beads; as the beads are approximately spherical, the total surface area of the beads is 4 times the surface area of the surface of the plate or culture vessel. Table 6 provides a range of bead concentrations for a 3.05 μm diameter polystyrene bead.

TABLE 6

| | | | Ratio of bead |
|---|---|---|---|
| | | Bead surface area | surface area to |
| | Bead concentration | per unit volume | culture vessel |
| Bead dose | (beads/mL) | (cm²/mL) | area |
| 0.02 | 1.00E+06 | 0.29210 | 0.08:1 |
| 0.05 | 2.70E+06 | 0.78867 | 0.2:1 |
| 0.1 | 5.40E+06 | 1.57733 | 0.4:1 |
| 0.25 | 1.35E+07 | 3.94333 | 1:1 |
| 0.5 | 2.70E+07 | 7.88666 | 2:1 |
| 1 | 5.40E+07 | 15.77332 | 4:1 |

The Notch signalling ligand density (such as, for example, the density of surface-bound DL4) on the bead may vary from, for example, 100 molecules per square micrometer (100 molecules/m²) to 3000 molecules per square micrometer (3000 molecules/μm²). Table 7 provides a calculation of the Notch signalling ligand concentration for a range of bead doses and Notch signalling ligand densities for 3.05 μm polystyrene beads.

TABLE 7

| | | | Notch ligand | Notch ligand | Notch ligand |
|---|---|---|---|---|---|
| | | Bead | concentration | concentration | concentration |
| | | surface | (molecules/mL | (molecules/mL | (molecules/mL |
| | | area | media) for bead | media) for bead | media) for bead |
| | Bead | per unit | with surface | with surface | with surface |
| Bead | concentration | volume | density of 100 | density of 800 | density of 3000 |
| dose | (beads/mL) | (cm²/mL) | molecules/μm² | molecules/μm² | molecules/μm² |
| 0.05 | 2.70E+06 | 0.78867 | 7.89E9 | 6.31E10 | 2.37E11 |
| 0.1 | 5.40E+06 | 1.57733 | 1.58E10 | 1.26E11 | 4.73E11 |
| 0.25 | 1.35E+07 | 3.94333 | 3.94E10 | 3.15E11 | 1.18E12 |
| 0.5 | 2.70E+07 | 7.88666 | 7.89E10 | 6.31E11 | 2.37E12 |
| 1 | 5.40E+07 | 15.77332 | 1.58E11 | 1.26E12 | 4.73E12 |

The 3D ETN may also comprise surface-bound VCAM-1. VCAM-1 may be immobilized to 3D ETN at an input molar ratio ranging from 1:6 to 10:1 DL4:VCAM-1. In an embodiment, the 3D ETN is prepared with an input molar ratio of 2.5:1 DL4:VCAM-1. In an embodiment, the final density of the surface-bound VCAM-1 on the bead is equivalent to the density of the Notch ligand. For example, the VCAM-1 surface area per unit volume may be equivalent to the Notch ligand surface area per unit volume shown in Table 7 above.

Cells may be cultured at densities appropriate for culture scale and format. In microplate culture cells may be cultured at, for example, 2.5×10⁵-2×10⁶ cells/mL. In STR culture cells may be cultured at, for example 5×10⁴-6×10⁶ cells/mL.

### Preparation of CD19 Antigen

It was first evaluated whether recombinant CD19 proteins, including the wild type and selected mutants, fulfill the criteria for being used for preparing cell stimulation/expansion reagents, specifically whether the recombinant protein could be functionally produced, and functionally immobilized.

While artificial antigen-presenting cells expressing the wild-type sequence of the ectodomain of CD19 (e.g. CD19-transduced Raji cells) have been used for stimulation and expansion of anti-CD19 CAR-T cells, the use of recombinant wtCD19 (ectodomain) in a defined system such as protein-functionalized beads has not been reported. Beads functionalized with anti-FMC63 scFv antibody have been shown to induce small to moderate expansion on FMC63-derived anti-CD19 CAR T cells (Philipson, 2020). Besides the low efficiency, the use of anti-idiotype antibody limits application to specific lines of CAR T cells (i.e. in this case, the FMC63-derived anti-CD19 CAR cells). Instead, antigen-coated beads may be applicable for all anti-CD19 CAR cells regardless of the CAR design.

The ectodomain of wild-type human CD19 was produced in varied designs using the HEK cell system following standard recombinant protein batch production conditions. Protein designs tested includes (1) full-length wtCD19 ectodomain (etCD19, Pro20-Lys291, AcroBiosystems) fused to human IgG1a Fc tag for improved solubility and a dimeric presentation; (2) etCD19 fused to human serum albumin domain 2 (AD2) for enhanced solubility; (3) truncated etCD19 (21-271) fused to human IgG1a Fc tag for improved solubility and a dimeric presentation; (4) etCD19 ectodomain fused to human IgG1a Fc tag with an artificial secretion signal peptide. Lobner et al. has reported recombinant preparation of homogeneous etCD19-AD2 using stably-transfected CHO-K1 cell line and an optimized cell culture process (specifically, with supplementation of chemical chaperone and use of semi-continuous perfusion culture process) (Lobner 2020). The authors also demonstrated the recombinant wtCD19-AD2, when immobilized on glass-supported bilayer lipid along with adhesion molecule ICAM1 and costimulatory molecule B7-1, activates CD19 CAR-T cells as evidenced by the triggering of intracellular calcium flux and formation of Supra-Molecular Activation Clusters at the contact (Lobner, 2020). Nonetheless, recombinant production of the above four designs including the etCD19-AD2 results in low titer, and the one-step purified proteins form higher-order oligomers.

The preparation of soluble functional protein is critical for the successful engineering of the antigen-functionalized T cell expansion reagents. Klesmith et al. has engineered several variants of the etCD19 with improved solubility using directed evolution (Klesmith, 2019). It was also previously demonstrated that the engineered etCD19 variants, when fused to scFv targeting cancer biomarkers such as CD20, can re-directed anti-CD19 CAR T to kill CD20-expressing cells (Klesmith, 2019). It was identified in the present disclosure that these engineered variants of etCD19, with their improved solubility, can be used for the preparation of functional reagents for CAR cell expansion. As an example, CD19.1, one of the engineered variants reported by Klesmith, was chosen and His-tagged and hFc tagged versions were produced in the HEK cell line using conventional batch culture conditions. Good titer was achieved for both versions. Sequences of wtCD19, CD19.1, and CD19.1-Fc are shown in Table 8 below.

TABLE 8

| Protein | Species | Sequence |
|---|---|---|
| etCD19 | Human | PEEPLVVKVEEGDNAVLQCLKGTSDGPTQQLTWSRESPLKPFLKLSLGLPGLGIHMRPLAI |
| (wtCD1 | | WLFIFNVSQQMGGFYLCQPGPPSEKAWQPGWTVNVEGSGELFRWNVSDLGGLGCGLK |
| 9) | | NRSSEGPSSPSGKLMSPKLYVWAKDRPEIWEGEPPCLPPRDSLNQSLSQDLTMAPGSTLW |
| | | LSCGVPPDSVSRGPLSWTHVHPKGPKSLLSLELKDDRPARDMWVMETGLLLPRATAQDA |
| | | GKYYCHRGNLTMSFHLEITARPVLWHWLLRTGGWK (SEQ ID NO: 6) |
| CD19.1 | Human | PEEPLVVKVEEGDEAWLPCLKGTSDGPTQQLTWSRESPLKPFLKVSFGVPGLGVHVRPNA |
| | | VSLVISNVSQQMGGFYLCQPGPPSEKAWQPGWTVNVEGSGELFRWNVSDLGGLGCGLK |
| | | NRSSEGPSSPSGKLMSPKLYVWAKDRPEIWEGEPPCLPPRDSLNQSLSRDMTVAPGSTLW |
| | | LSCGVPPDSVSRGPLSWTHVHPKGPKSLLSLELKDDRPARDMWVTGTRLFLPRATAQDAG |
| | | KYYCHRGNLTMSFHLEVKARPVSAHTKLRTGGWK (SEQ ID NO: 7) |
| CD19.1- | Human | MPPPRLLFFLLFLTPMEVRPEEPLVVKVEEGDEAWLPCLKGTSDGPTQQLTWSRESPLKPFL |
| Fc | | KVSFGVPGLGVHVRPNAVSLVISNVSQQMGGFYLCQPGPPSEKAWQPGWTVNVEGSGE |
| | | LFRWNVSDLGGLGCGLKNRSSEGPSSPSGKLMSPKLYVWAKDRPEIWEGEPPCLPPRDSL |
| | | NQSLSRDMTVAPGSTLWLSCGVPPDSVSRGPLSWTHVHPKGPKSLLSLELKDDRPARDM |
| | | WVTGTRLFLPRATAQDAGKYYCHRGNLTMSFHLEVKARPVSAHTKLRTGGWKGGGSGG |
| | | GSPKSSDKTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK (SEQ ID NO: |
| | | 8) |

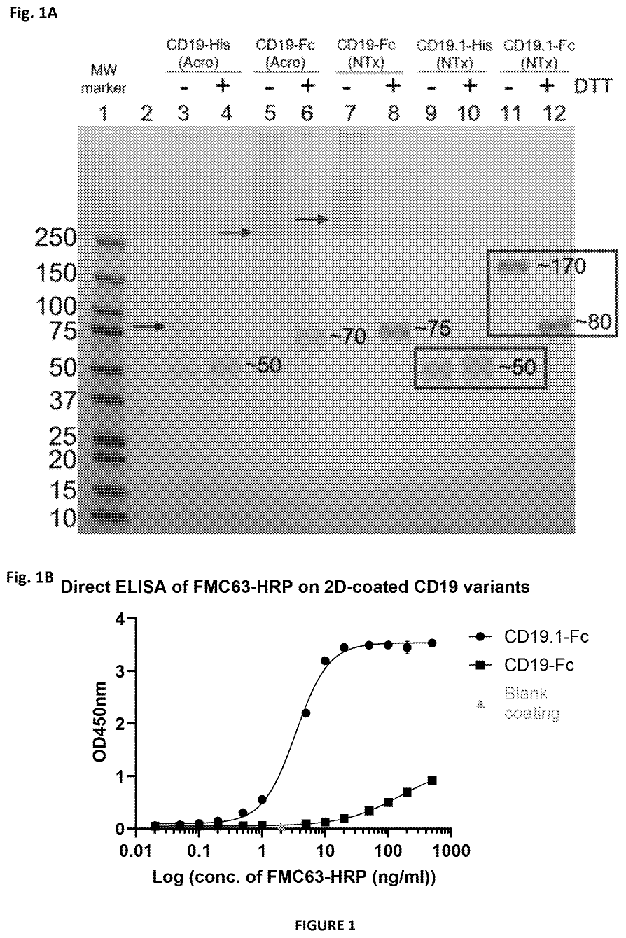

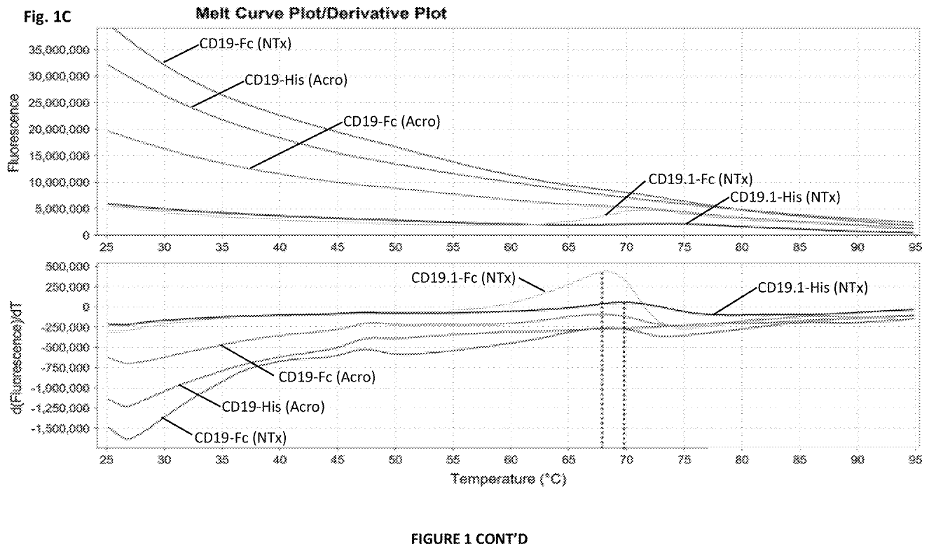

The solubility of the etCD19 variants was compared using SDS-PAGE under non-reducing conditions. Both the His-tagged and the hFc-tagged CD19.1 migrate as a single band matching their theoretical molecular weight under non-reducing conditions (FIG. 1A), whereas the commercial (AcroBiosystems) and custom produced wild type etCD19 proteins (lane 3, 5, 7) formed high-order oligomers (FIG. 1A). A direct ELISA of the CD19 antibody FMC63 on 2D-coated CD19-Fc and CD19.1-Fc demonstrated that CD19.1 was superior to wtCD19 in binding to FMC63 (FIG. 1B). Furthermore, the thermal stability of the etCD19 variants was characterized using a protein thermal shift assay (GloMelt, Biotium). CD19.1-His and CD19.1-Fc in PBS displayed a normal thermal unfolding profile with denaturing midpoints of 69.9° C. and 68.3° C., respectively (FIG. 1C). In contrast, the thermal unfolding profile of WT ecCD19 either purchased from a reputable vendor (AcroBiosystems) or custom-produced, did not show any detectable unfolding transition (FIG. 1C). Moreover, the WT ecCD19 exhibited high fluorescence at room temperature, suggesting the protein was in a partially unfolded state or/and highly hydrophobic (FIG. 1C).

The binding affinity of the CD19 variants to immobilized FMC63 was also characterized using biolayer interferometry. The equilibrium dissociation constants (K_{D}) of CD19.1-Fc and CD19.1-His were 0.103 nM and 3.18 nM respectively. The custom-produced WT etCD19-Fc displayed strong non-specific binding to the blank sensor surface and weak binding to the FMC63 surface, suggesting the protein formed inactive soluble aggregate. In comparison, K_{D} values for the purchased WT etCD19-His and WT etCD19-His (Acrobiosystems) are 3.88 nM and 7.18 nM respectively. However, a stronger non-specific binding is observed for these proteins.

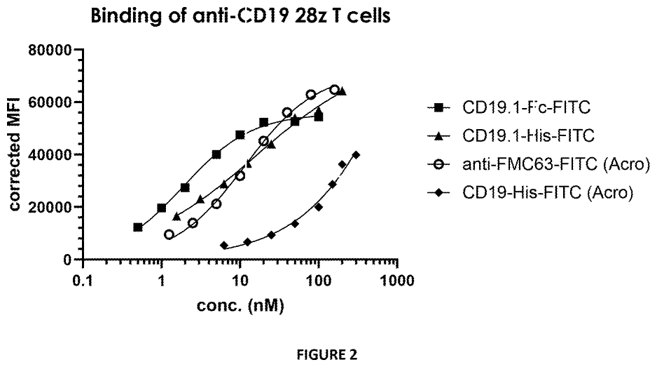

The specific binding of CD19.1 (both -His and -hFc tagged versions) to anti-CD19 CAR cells was assessed by titrating FMC63-derived CAR cells with FITC-labeled CD19.1. EC50 was determined by fitting the binding curve with a four-parameter dose-response function. When titrated on anti-CD19 28z CAR primary T cells, the EC50 values for CD19.1-Fc and CD19.1-His were 1.9 nM and 15.8 nM, respectively (FIG. 2). In comparison, the binding curve of the commercially available FITC-CD19 (AcroBiosystems) to the same CAR cells was shifted to a much higher concentration (FIG. 2). Therefore, the engineered CD19.1 demonstrated a much stronger binding strength to anti-CD19 CAR cells than wtCD19.

### Preparation of CD19 Antigen Beads

Smooth, carboxyl group functionalized, magnetic polystyrene beads of 4.5 μm were used to conjugate the CD19.1 protein using EDC/NHC conjugation as described below.

Beads were resuspended in the vial by gentle vortexing. 200 μL of beads were transferred to a low protein binding Eppendorf tube. 200 μL of activation buffer was added and mixed thoroughly, and the supernatant was decanted. These steps were repeated twice. After the final wash, the beads were resuspended in 200 μL of activation buffer. Freshly prepared EDC and NHS were added quickly to 200 μL of washed beads and incubated by mixing on a rotator for 30 minutes at room temperature. The tube was placed in a magnet for 1 min and the supernatant was discarded. The tube was removed from the magnet and the washed beads were resuspended in the same volume of conjugation buffer as the initial volume of beads (i.e. 200 μL) and mixed thoroughly. This washing step was repeated twice. The Eppendorf tube was placed in a magnet and the volume of protein to be added was carefully removed (i.e., 200 μL minus the volume of protein). Based on the total moles of carboxyl groups available for protein conjugation on the beads, 0.5× moles, 1.0× moles, 2.0× moles, and 5.0× moles of CD19.1 protein were added, respectively, into the reaction mixture. The total conjugation reaction volume was 200 μL. The reaction was allowed to proceed for 2.5 hours at room temperature with gentle tilting and rotation. Glycine was added to quench the reaction and incubated at room temperature for 30 minutes with gentle mixing. A magnet was applied for 1 minute, and the supernatant was carefully transferred into a clean Eppendorf tube. Blocking buffer was used to wash three times. The beads were resuspended in 200 μL of blocking buffer and mixed for a minimum of 2 hours. A magnet was applied for 1 minute and the supernatant was discarded and replaced with the same volume of storage buffer. 200 μL of storage buffer was added and the coated beads were stored at 2° C. to 8° C. until further use.

The density of CD19.1 protein on beads was determined by performing a bicinchoninic assay (BCA) directly on CD19.1 coated beads. The coating efficiency as reported by percentage of the theoretical maximum coverage for each condition is reported in Table 9 below.

TABLE 9

| Coated Bead | % of Theoretical maximum coverage |
|---|---|
| CD19.1-Fc (0.5 × moles) | 45.24 |
| CD19.1-Fc (1.0 × moles) | 51.67 |
| CD19.1-Fc (2.0 × moles) | 55.00 |
| CD19.1-Fc (5.0 × moles) | 59.86 |
| CD19.1-His (0.5 × moles) | 43.34 |
| CD19.1-His (1.0 × moles) | 51.40 |
| CD19.1-His (2.0 × moles) | 51.41 |
| CD19.1-His (5.0 × moles) | 69.4 |

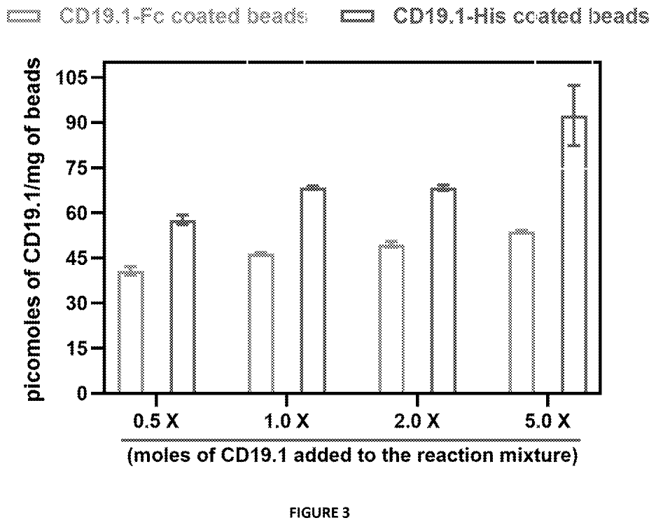

As expected, a maximum CD19.1 protein loading on beads was observed when the protein concentration was the highest in the reaction mixture i.e., 5.0× moles (FIG. 3).

The binding capacity of CD19.1 antigen-coated beads was determined by titrating beads with an excess amount of anti-CD19-FITC (FMC63 clone). The amount of anti-CD19-FITC bound to the beads was then interpolated from the standard curve generated with anti-CD19-FITC. Briefly, 20 μL of uncoated/coated bead was incubated for 60 minutes with anti-CD19-FITC (FMC63) at room temperature with gentle mixing. The magnet was applied, and the supernatant carefully removed. The beads were washed three times with the buffer. The fluorescence intensity of supernatants and washings were analyzed. The washed beads were resuspended in 500 μL of buffer, 200 μL was transferred into wells of a black 96-well plate, and the fluorescence intensity was measured. Data shows that higher anti-CD19 binding can be achieved by increasing the antigen density (Table 10 below). This demonstrates the ability to control antigen loading and modulate cell activation.

TABLE 10

| | % of CD19.1 | |
|---|---|---|
| | molecules bound | |
| | by anti-CD19-FITC | Moles of antibody |
| Coated Beads | (FM63-FITC) | per mg of beads |
| CD19.1-Fc (0.5 × moles) | 0.70 | 0.28E−12 |
| CD19.1-Fc (1.0 × moles) | 0.87 | 0.41E−12 |
| CD19.1-Fc (2.0 × moles) | 1.21 | 0.6E−12 |
| CD19.1-Fc (5.0 × moles) | 1.86 | 1.00E−12 |
| CD19.1-His (0.5 × moles) | 0.80 | 0.49E−12 |
| CD19.1-His (1.0 × moles) | 0.76 | 0.51E−12 |
| CD19.1-His (2.0 × moles) | 1.66 | 1.14E−12 |
| CD19.1-His (5.0 × moles) | 2.17 | 2.00E−12 |

### Example 2: Culture of CAR T Cells with CD19 Coated Beads

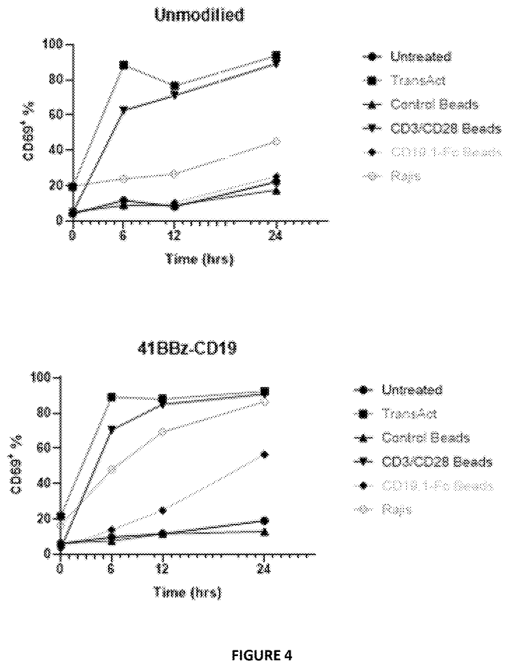

CART cells were co-cultured with CD19.1Fc-coated beads at a 1:1 ratio, and CD69 expression was measured by flow cytometry at increasing time points to assess T cell activation. Human primary CAR T cells were plated in U-bottom plates and equal amounts of media containing activation reagents (TransAct™ (Miltenyi), CD3/CD28 beads, CD19.1-FC beads) or controls (uncoated beads, Raji target cells) were added. Cells were cultured over time; and, at indicated time points, CAR T cells were stained for viability, fixed, and stained with a T cell activation flow panel. CD19.1-Fc-coated beads demonstrated specific activation of CAR+ primary T cells in comparison with unmodified primary T cells (FIG. 4).

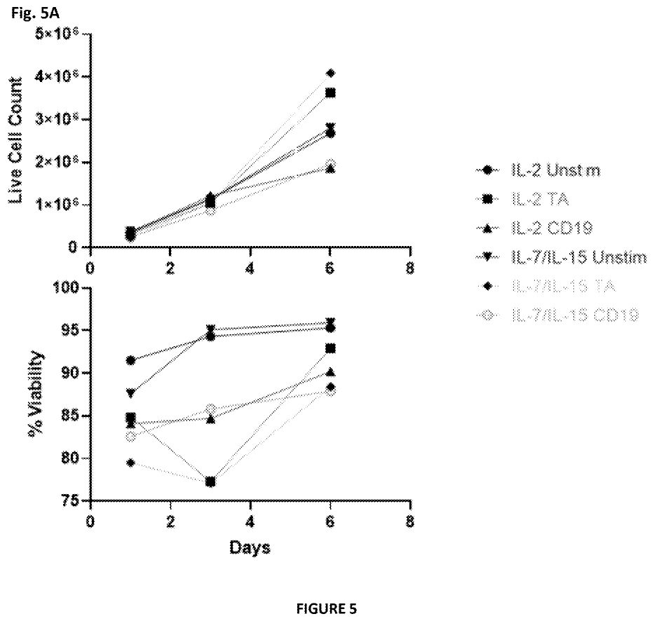

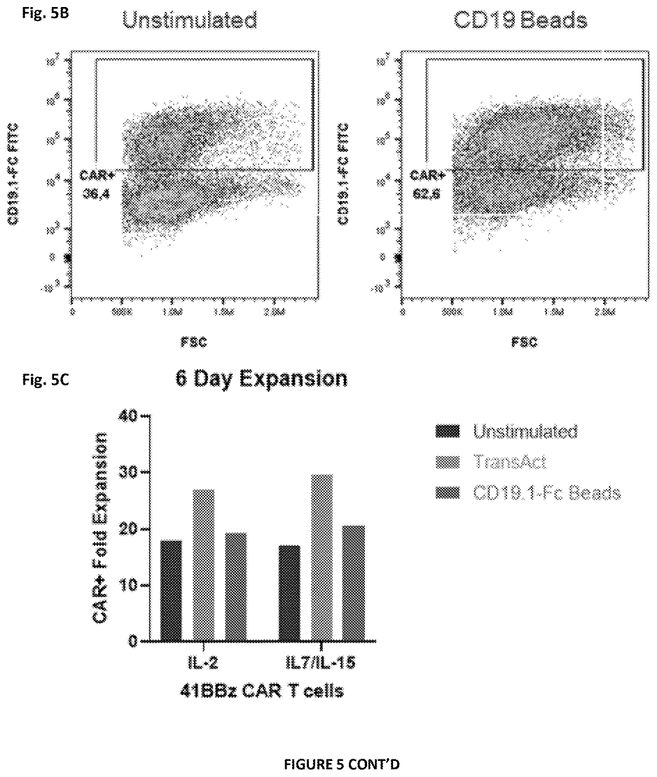

For expansion, the CAR T cells were cultured in X-VIVO™ 15 media with (1) IL-2 (200 U/mL), or (2) IL-7 (10 ng/mL) plus IL-15 (10 ng/mL) in tissue culture plates for about one week. The CAR-T cells were incubated at 37° C. with 5% CO2, and the culture medium and cytokines were replaced every two days. Viability and cell counts were measured with the Via2 cassette on a Nucleocounter. For CAR T cell enrichment assessment, cells were stained for viability and CAR detection with a CAR-specific monoclonal antibody. CAR-T cells expanded and improved viability over time with CD19-coated beads in the presence of IL-2 or IL7 and IL-15 (FIG. 5A, B). CD19 bead stimulation resulted in enriched CAR expression (FIG. 5C).

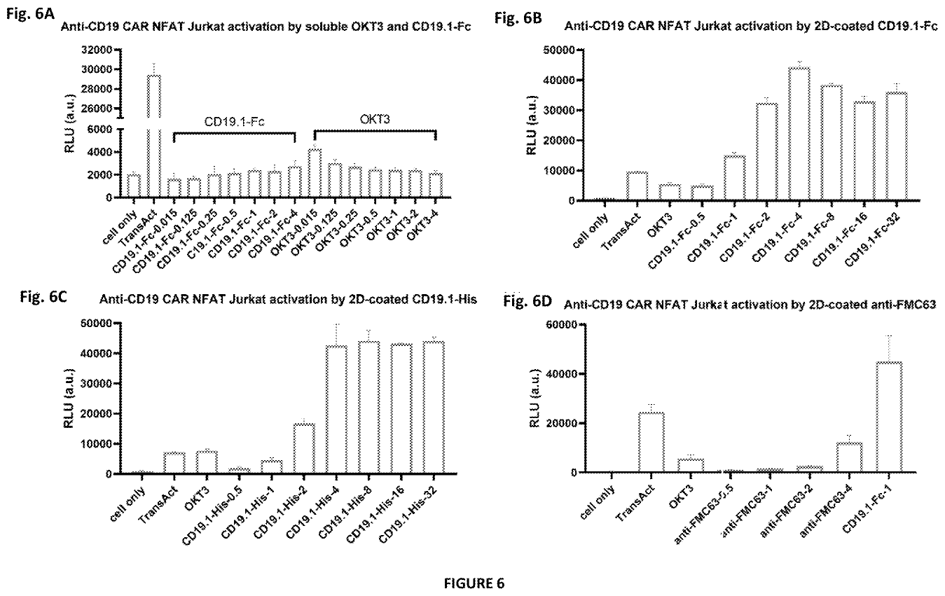

To verify the activity of CD19.1 for CAR T cell activation, a luciferase reporter cell line was developed by transducing the Jurkat-Lucia NFAT cells (Invivogen) with FMC63-scFv-41BBz CAR. Upon stimulus with treatments such as PMA and ionomycin, the Jurkat-Lucia NFAT cells turned on the expression of Lucia luciferase (data not shown). Modified CAR Jurkat-Lucia NFAT cell line responded to conventional T cell stimulation reagents such as TransAct (Miltenyi) and anti-CD3/anti-CD28 Dynabeads (ThermoFisher) (FIG. 6A) No luciferase activity or T cell activation was observed when the CD19.1 (either the -His or the -hFc tagged versions) were added in soluble form (FIG. 6A). CD19.1 was then immobilized on 96-well tissue culture plates by passive adsorption. Specifically, 100 μL of the protein in PBS was added to 96-well plates and incubated at 37° for 2 hr. When the CAR Jurkat NFAT cells were seeded on the CD19-coated plate, a dramatic increase in the luciferase activity was observed (FIG. 6B, C). Activation caused by both proteins was dose-dependent (FIG. 6B, C). Although CD19.1-Fc appeared to have a lower EC50 and thus more potent than CD19.1-His, the maximum stimulation was comparable between the two. The lower EC50 observed for CD19.1-His may be caused by lower coating efficiency given its smaller size. It was explicitly demonstrated that although the soluble CD19.1 binds to anti-CD19 CAR T cells (see example 1.1B), immobilization is required for its T cell activation (FIG. 6A vs. 6B, C). Immobilized CD19.1 was highly potent in CAR T cell activation.

As a comparison, a commercially available anti-FMC63 scFv (clone FM3-Y45, AcroBiosystems) was also immobilized and its potential for T cell activation investigated. The clone FM3-Y45 also activated the reporter cells in a dose-dependent manner (FIG. 6D). However, it was less potent and activation strength was >30-times lower than CD19.1 when coated at the same mass density (FIG. 6D). It should be noted that the maximum stimulation caused by the immobilized CD19.1 was 3.4-fold stronger than that by TransAct™ added at the manufacturer's recommended dose (i.e., 1:100 dilution). Although stronger activation is not necessarily beneficial for T cell expansion or killing activity, it implies a broader dynamic range which in turn allows more freedom for tuning.

To evaluate the functional activity of CD19 coated beads, 41BB-NFAT-Jurkat reporter cell lines were treated with the CD19 coated beads. In a U-bottom 96-well plate, 5E4 cells/well were seeded in 50 μL of media. Then 50 μL of media was added containing CD19 coated beads, BSA coated beads, or conventional T cell activating beads (CD3/CD28 Dynabeads, or TransAct™ human T cell activator). At each time point, 20 μL of supernatant was removed and 50 μL of reconstituted QUANTI-Luc Gold (luciferase substrate) reagent was added into each well. The luminescence was then measured using a spectrophotometer.

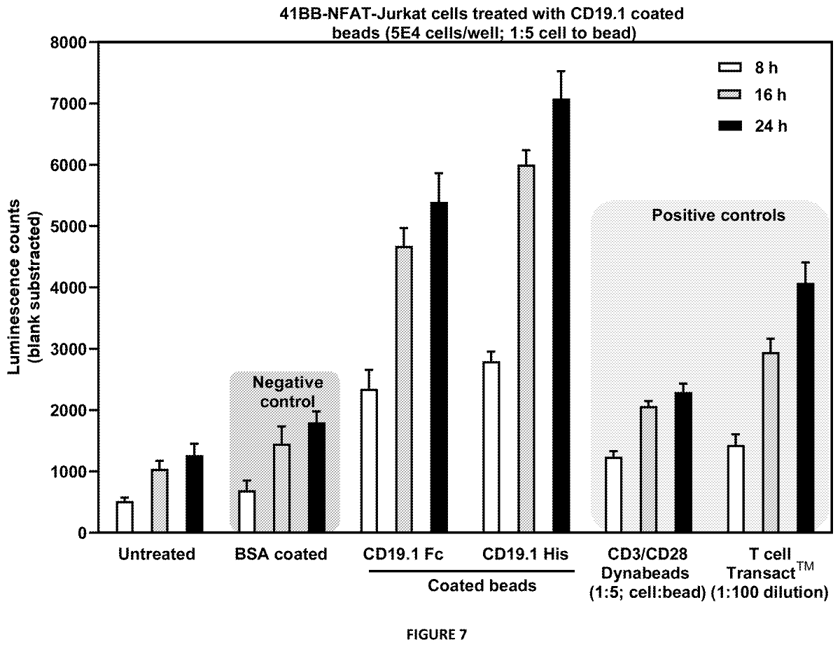

The activation of cells by CD19.1 coated beads was higher than the conventional CD3/CD28 Dynabeads and the soluble T-cell activator TransAct™ (FIG. 7).

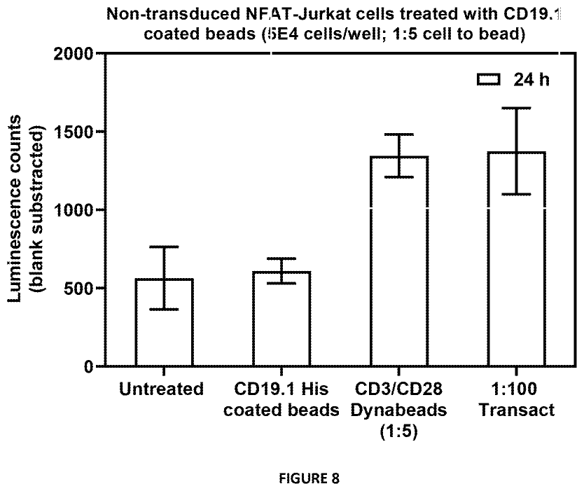

Next, it was evaluated whether CD19.1 coated beads would show specificity towards CD19 CAR T cells. Non-transduced NFAT-Jurkat cells were incubated with CD19.1 coated beads. No activation of cells was observed with CD19.1 coated beads, while non-specific stimulation was observed with the CD3/CD28 Dynabeads™ and the soluble T-cell activator TransAct™ (FIG. 8).

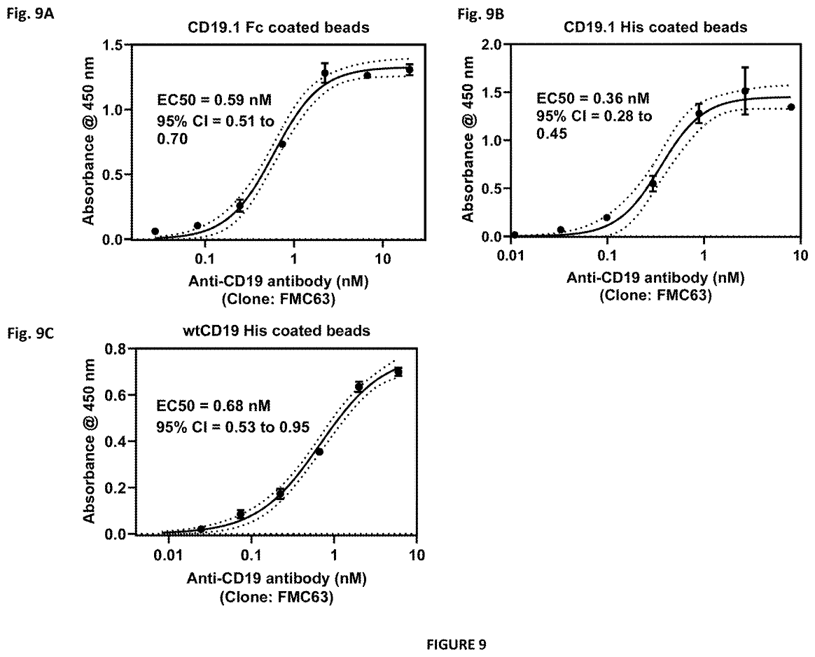

An ELISA was performed to determine the half-maximal effective concentration (EC50) values of CD19-coated beads (CD19.1-Fc, CD19.1-His, and wtCD19-His). PBS containing BSA (0.1% w/v) buffer (pH=7.4) was added to all wells and incubated for 5 minutes at room temperature. The CD19.1-coated bead solution was added into wells of low binding flat bottom, 96-well plate, with uncoated beads as controls (+/−antibody) and blanks. A DynaMag™ side-skirted magnet was applied to pull beads to the side of the plate and the supernatant removed. Then 100 μL HRP conjugated anti-human CD19 (FMC63) antibody was added to each well. The plate was sealed and incubated for 1 hour at room temperature with mixing. The DynaMag™ side-skirted magnet was applied to pull beads to the side of the plate and the supernatant removed. The plate was washed three times with PBS containing 0.05% v/v Tween-20, and the side-skirted magnet was applied to remove the supernatant. 100 μL of 1-step Ultra TMB-ELISA (ThermoFisher) solution was added, and the plate was covered and incubated at room temperature in dark conditions for 30 minutes or until the desired color was developed. The reaction was stopped by adding 100 μL 2M sulfuric acid. The DynaMag™ side-skirted magnet was applied to pull beads to the side of the plate and 100 μL was pipetted from each well and transferred into another 96-well plate to measure the absorbance. The plate was evaluated within 30 minutes of stopping the reaction. The absorbance of each well was read at 450 nm and 550 nm, and the 550 nm values were subtracted from the 450 nm values. Curves were fit using a four-parameter logistic regression to calculate the EC50. CD19.1-His-coated beads were observed to have the lowest EC50 compared to CD19.1-Fc and wtCD19-His (FIG. 9A, B, C).

Both wtCD19 and CD19.1Fc were coated onto beads by adding the same amount of protein into the reaction mixture. The total protein density on wtCD19 and CD19.1Fc-coated beads was comparable (Table 11). An on-bead anti-CD19 binding assay was performed as described above, and it was observed that the binding capacity (functional antigen density) of CD19.1Fc coated beads was approximately 3.5 times higher than the wtCD19 coated beads (Table 11).

TABLE 11

| | Moles of | | |
|---|---|---|---|
| | protein | Percentage of CD19.1 or wt | Moles of |
| Coated | per mg | CD19 Fc molecules bound | antibody per |
| beads | of beads | by anti-CD19-FITC (FMC63) | mg of beads |
| CD19.1−Fc | 34.3E−12 | 1.36 | 0.46E−12 |
| wtCD19 Fc | 36.0E−12 | 0.37 | 0.13E−12 |

### Example 3: Culture of iPSC-Derived T Cell Progenitors and T Cells with CD19 Coated Beads

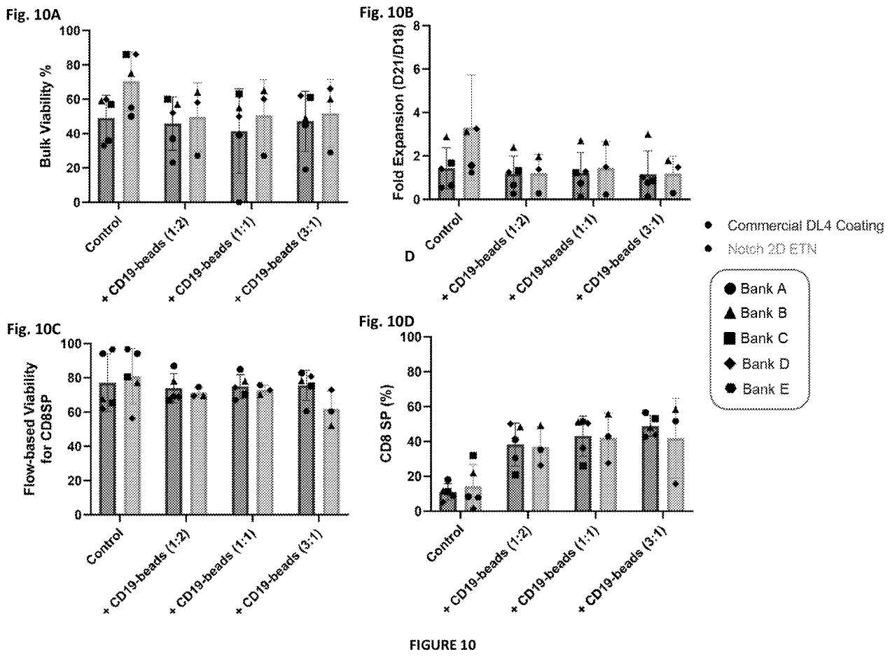

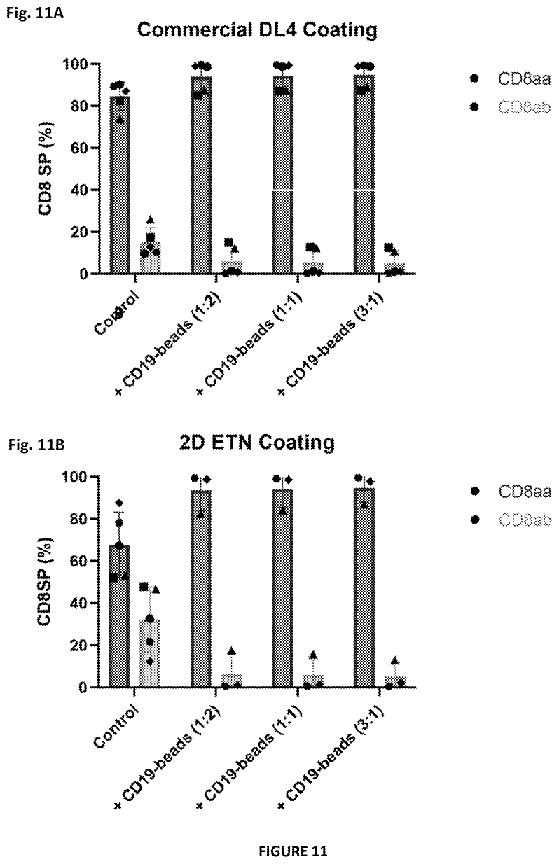

In this example, maturation of iPSC-derived polyclonal TRAC+/+ CAR-28z proT cells (Banks A-E) was performed by culture for 3 days with CD19 antigen beads (days 18-21 of maturation on commercial DL4 coating or 2D DL4+VCAM ETN). Cells were analyzed for viability, fold expansion, and marker expression. Bulk viability was maintained at approximately 50% across conditions (FIG. 10A) and fold expansion was consistent across conditions (FIG. 10B). CAR stimulation with CD19-coated beads increased viability of the CD4−CD8+ (CD8SP) population (FIG. 10C) and increased % CD8SP (FIG. 10D). Culture on commercial DL4 coating (StemSpan™ Lymphoid Differentiation Coating Material, STEMCELL Technologies) with and without CD19 beads predominantly generated CD8αα+ cells (FIG. 11A). Culture on 2D ETN (DL4 & VCAM) generated a mixed population of CD8αα+ and CD8α(3+ cells, and the percentage of CD8αβ+ cells decreased with CD19 bead stimulation (FIG. 11B).

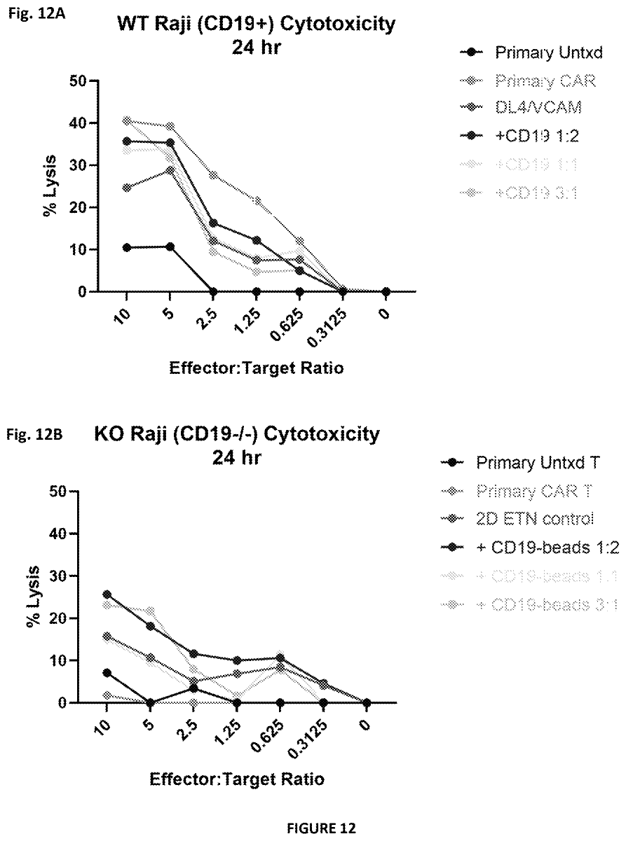

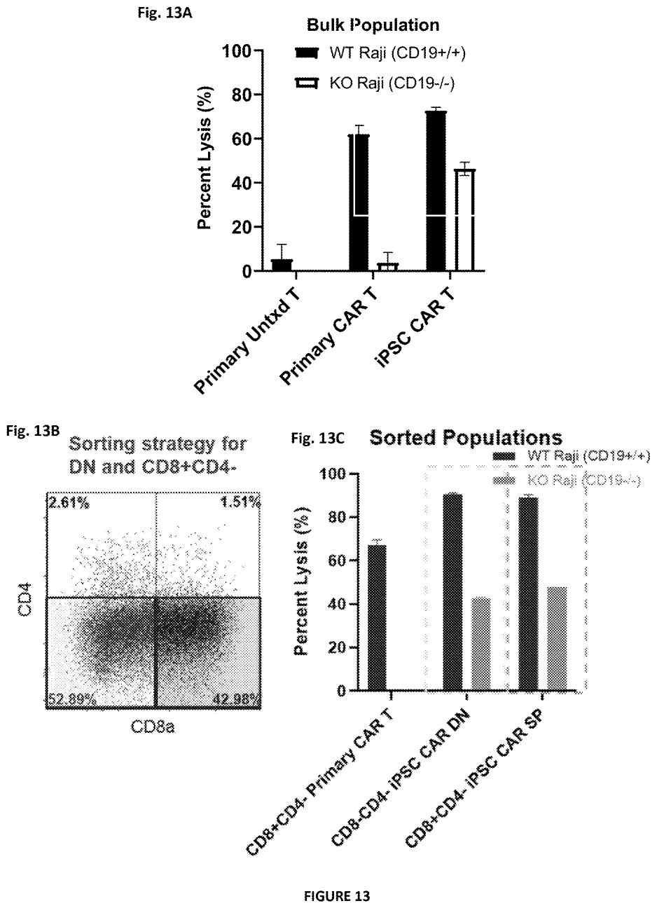

In a co-culture assay, iPSC-derived cells showed cytotoxicity against WT Raji comparable to primary T cells (FIG. 12A). Non-specific killing was also seen against CD19 KO Raji cells (FIG. 12B). This may have been due to innate-like properties of generated CD8 αα+ cells. In the co-culture assay, overall cell lysis was similar for iPSC CAR T and primary CAR T cells against WT CD19+ Raji cells (FIG. 13A). To investigate the contribution of sub-populations of iPSC-derived cells, CD4−CD8α− (DN) and CD4−CD8α+ cells were sorted (FIG. 13B) and co-cultured with target cells. Both DN and CD4−CD8α+ cells contributed to killing of WT and CD19 KO Raji (FIG. 13C).

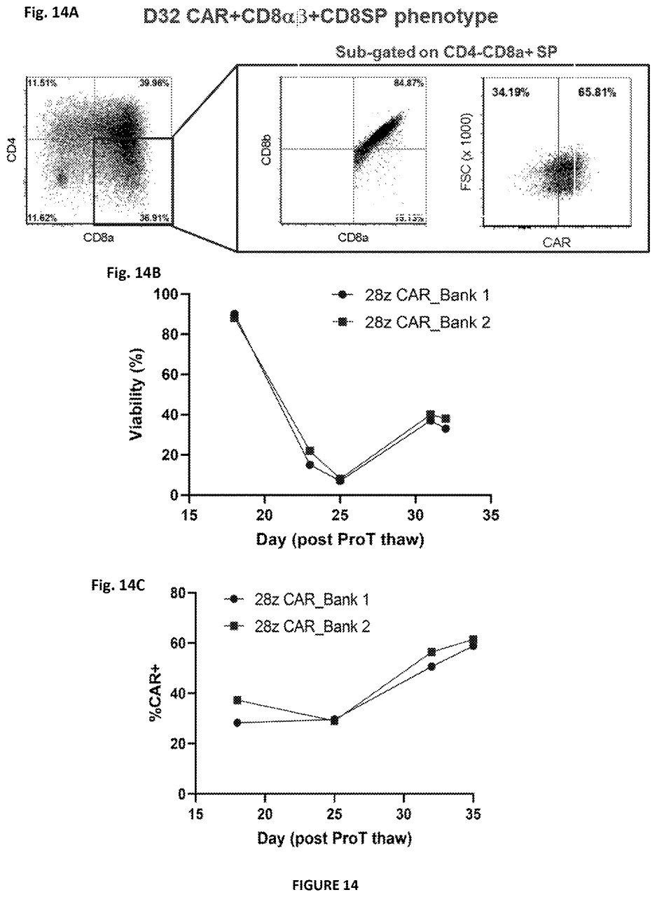

In a further experiment, CAR-modified iPSC-derived progenitor T cells (day 18 of differentiation from HSPCs) were cultured on 2D ETN with frequent re-feeding and cell marker expression was assessed at 14 days (FIG. 14A). Cell viability and CAR expression were assessed throughout the culture period (FIG. 14B, C).

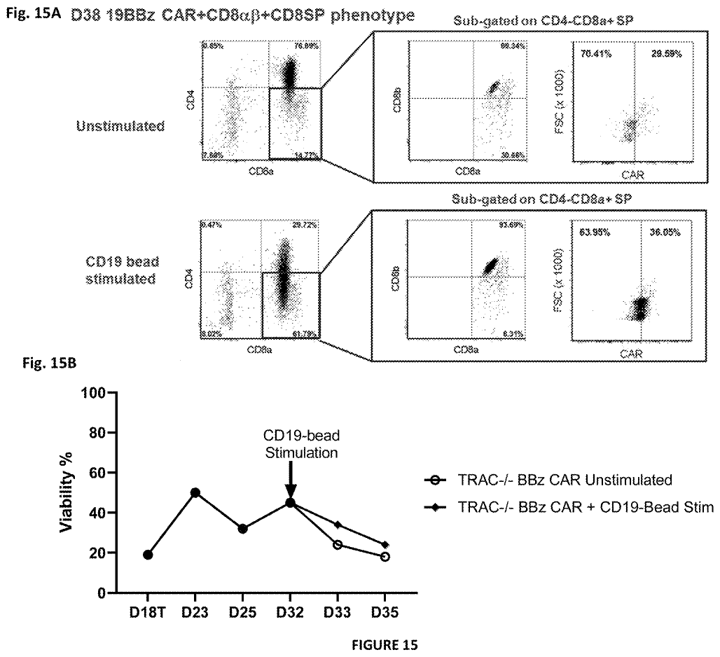

After 14 days, CD19 antigen bead stimulation was provided for 24 hours at a 1:2 bead:cell ratio. Cells were de-beaded and assessed after 2 days. Extended maturation time and antigen-dependent stimulation improved purity of CAR+ CD4−CD8αβ+ CD8 cells (FIG. 15A). Viability was also assessed pre- and post-stimulation with CD19 antigen-coated beads (FIG. 15B).

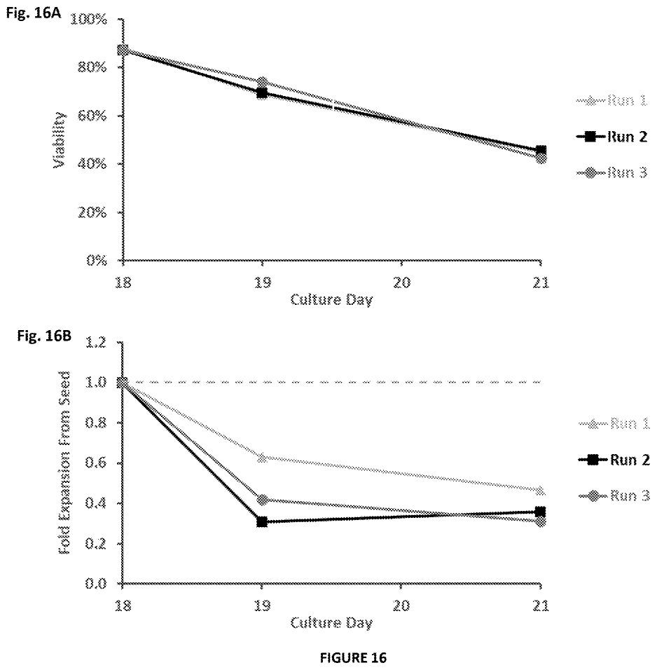

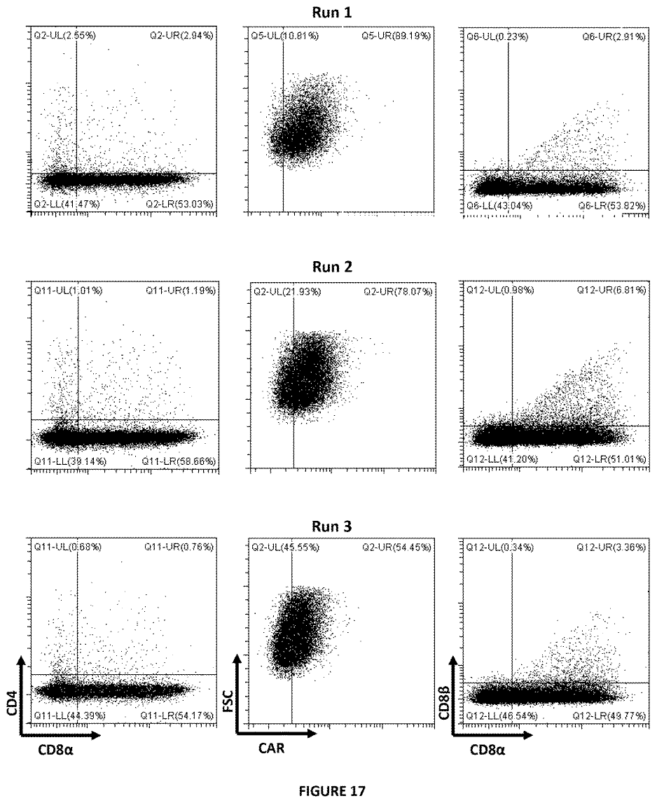

The above culture protocol was repeated for three independent runs of iPSC-derived progenitor T cells. Comparable viability and fold expansion was observed across runs (FIG. 16A, B). Table 12 provides the output results demonstrating that culture with CD19 antigen-coated beads resulted in generation of CD8α+ CAR+ cells (FIG. 17).

TABLE 12

| Parameter | Method | Run 1 | Run 2 | Run 3 |
|---|---|---|---|---|
| Bulk Viability | Cellometer | 45% | 46% | 43% |
| CD8α+ CD4− | Flow cytometry | 53% | 59% | 54% |
| CD8α+ CD4− CAR+ | Flow cytometry | 89% | 78% | 54% |

### Example 4: In Vitro Function of iPSC-Derived CD8αα Cells

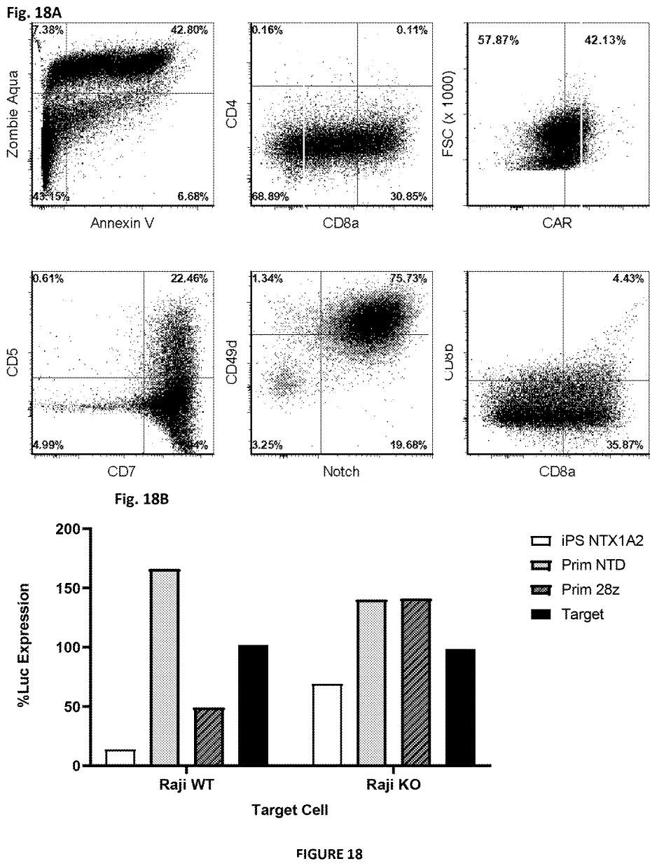

The in vitro function of CAR-modified iPSC-derived cells, generated as described above, was evaluated. Generated cells included CD4−CD8− DN and CD8αα+ phenotypes, as determined by flow cytometry (FIG. 18A). CAR-modified iPSC-derived cells demonstrated effective killing of CD19+ Raji wild-type cells and non-specific killing of CD19− KO Raji cells (FIG. 18B, “iPS NTX1A2”).

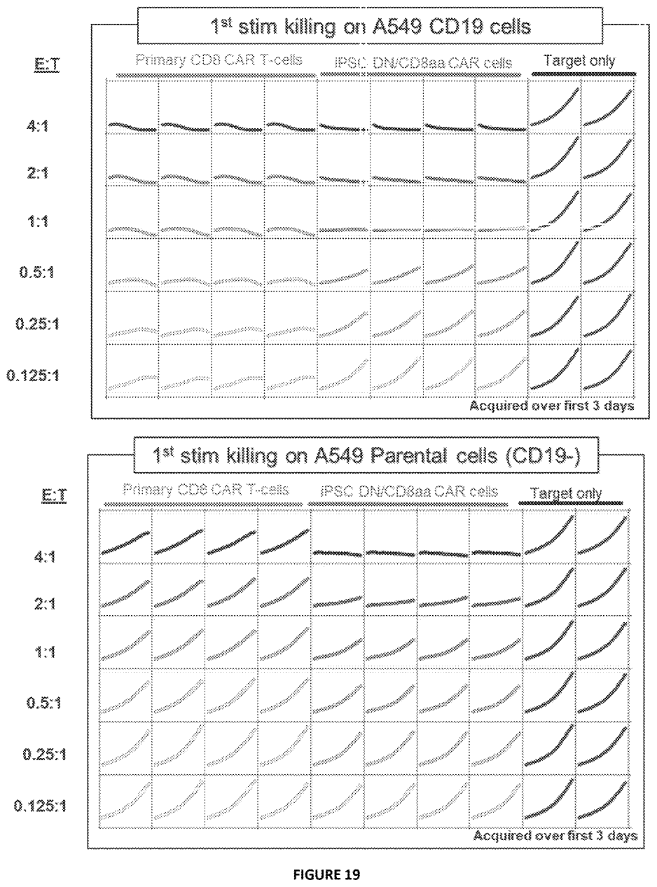

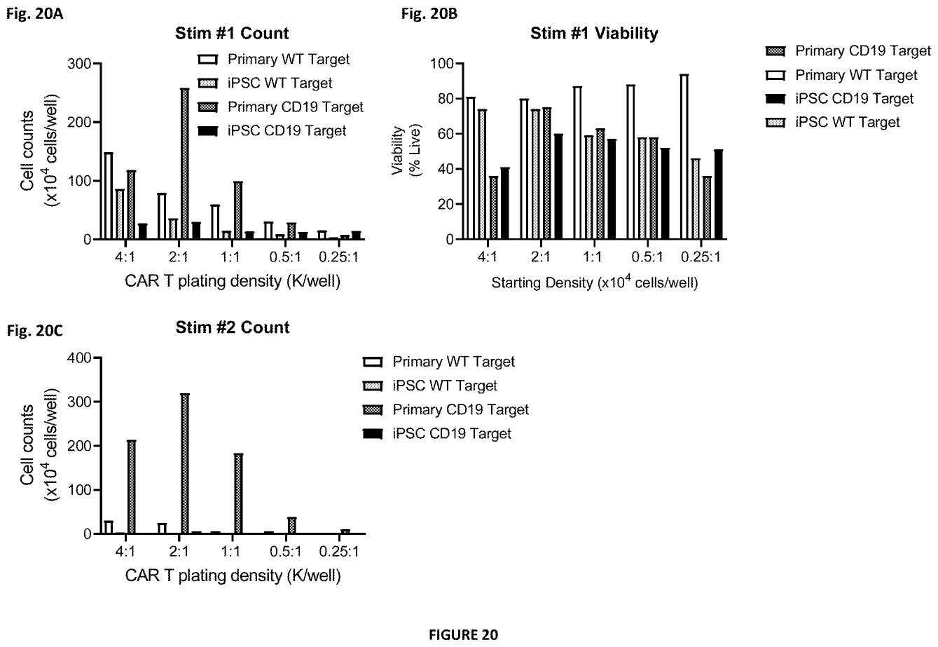

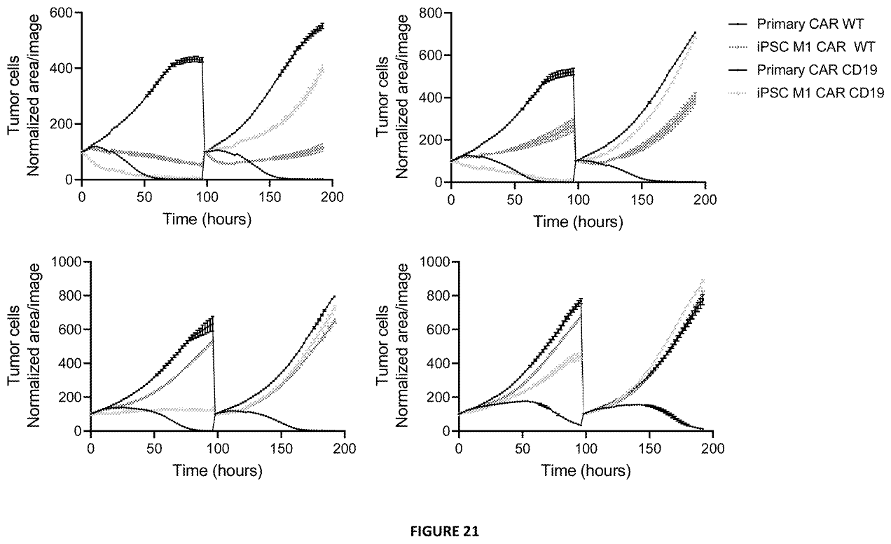

In a serial restimulation assay, primary CD8 CAR T cells (donor 3215) or iPSC-derived DN/CD8αα+ cells were co-cultured with CD19 overexpressing A549 cells or the CD19 negative A549 parental cell line. A549 lines expressed GFP. Each well was imaged for GFP positive area for 3 days, with 4 replicate wells for each E:T condition. iPSC-derived DN/CD8αα cells demonstrated specific and non-specific killing during the first stimulation (FIG. 19). Primary CAR T cells were functional in a 3rd stimulation; despite comparable viability, iPSC-derived CARs had insufficient counts for continuation beyond two stimulations (FIG. 20A-C). Over two rounds of co-culture at varying E:T ratios, iPSC-derived DN/CD8αα cells demonstrated specific and non-specific killing during the first stimulation and potency was lost in the second stimulation (FIG. 21).

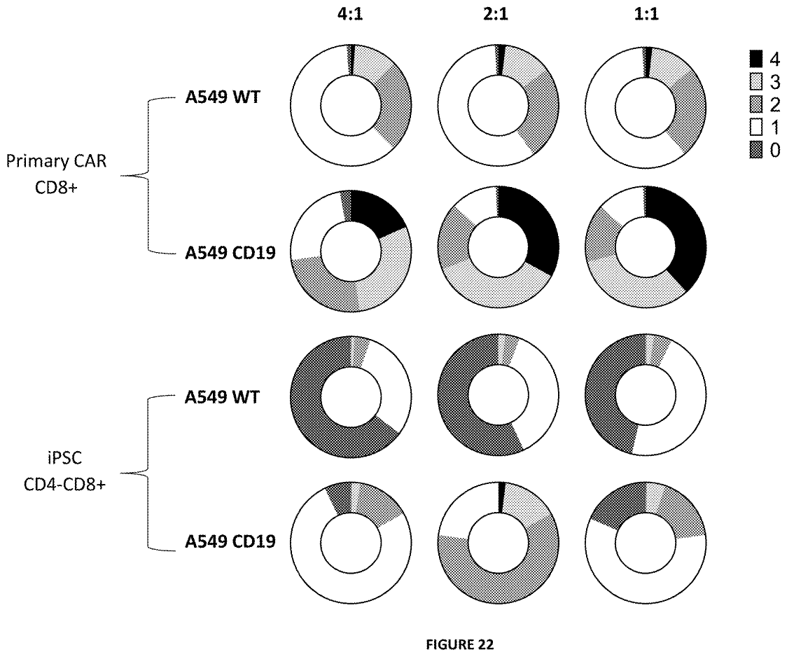

Expression of exhaustion markers TIGIT, TIM3, LAG3, PD-1 was evaluated for primary and iPSC-derived cells following the first stimulation with target cells. Classic exhaustion markers were not expressed in iPSC-derived CD8αα+ cells after the first stimulation, unlike primary CAR-T cells (FIG. 22).

### Example 5: Culture of T Cell Progenitors with CD19-Coated Beads and ETN Beads

In this example, PSC-derived T cell progenitors were cultured in the presence of CD19 antigen-coated beads in combination with Engineered Thymic Niche (ETN) beads. Day 18 progenitor cells were cultured on plate-bound 2D ETN, or with ETN beads at doses of 0.5× and 0.05× relative bead dose. All conditions were cultured in both the presence and absence of CD19 beads for 24 hours. After 24 hours, conditions cultured with CD19 beads were de-beaded and reseeded either onto fresh 2D ETN or with fresh ETN beads. Cells were assessed for viability and phenotypically by flow cytometry at the time of seeding (D18), after 24 hours of CD19 antigen bead stimulation (D19), and 2 days post-reseeding (D21).

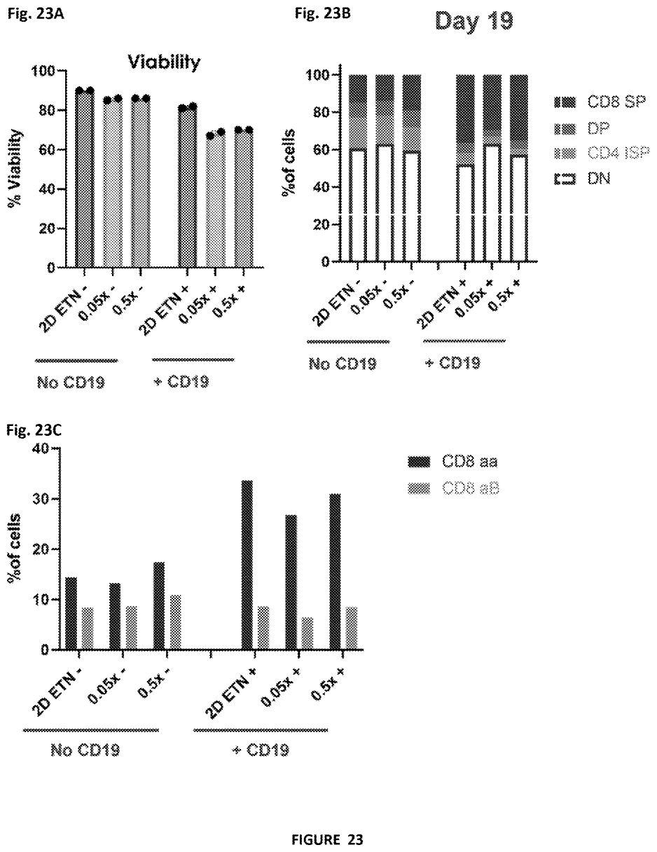

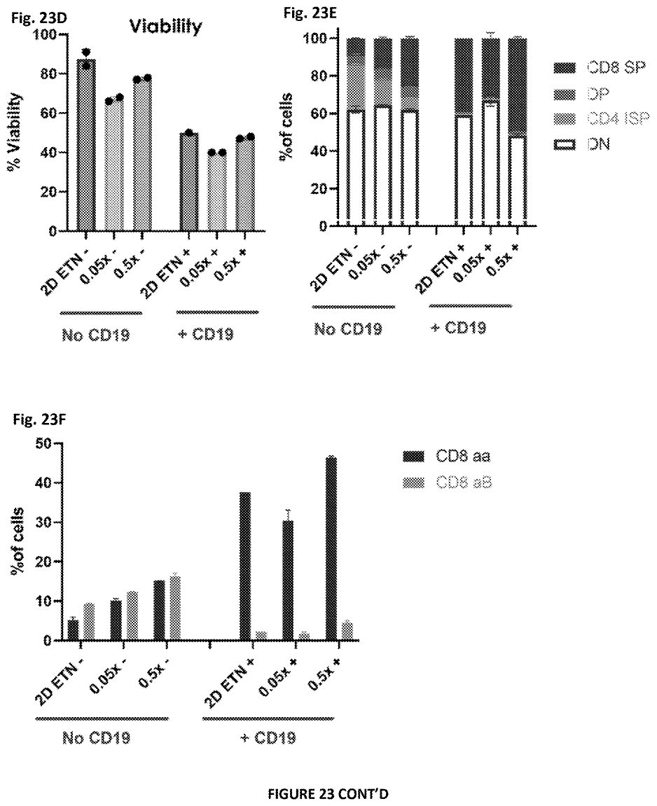

After 24 hours of bead stimulation (Day 19) cell phenotype was similar with 2D ETN and 0.5×3D ETN dose (FIG. 23B, Table 13). Viability was slightly higher for 2D ETN compared to 3D at day 19 (FIG. 23A). After 48 hours of recovery (Day 21), viability was similar between 2D ETN and 0.5×ETN bead dose, while 0.5×ETN bead dose displayed a higher proportion of CD4−CD8+ (CD8 SP) cells (FIG. 23D,E, Table 13).

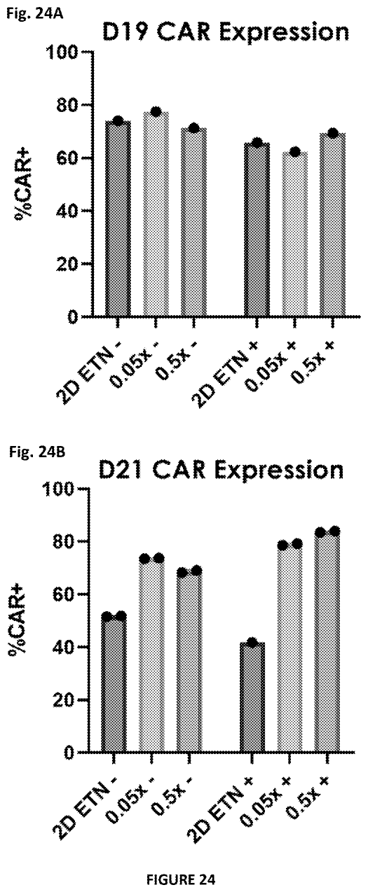

CAR expression was also higher in conditions cultured with ETN beads compared to 2D ETN at day 21 (FIG. 24, Table 13).

TABLE 7

| | | | 3D ETN | 3D ETN |
|---|---|---|---|---|
| Parameter | Method | 2D ETN | 0.5x | 0.05x |
| Bulk Viability | Cellometer | 50% | 47.5% | 40% |
| CD8α+ CD4− | Flow cytometry | 39.3% | 49.5% | 31.5% |
| CD8α+ CD4− | Flow cytometry | 47.5% | 88% | 82.5% |
| CAR+ | | | | |

### Example 6: Generation of iPSC-derived CD4−CD8+ Cells Via a Multi-Stage Differentiation Protocol

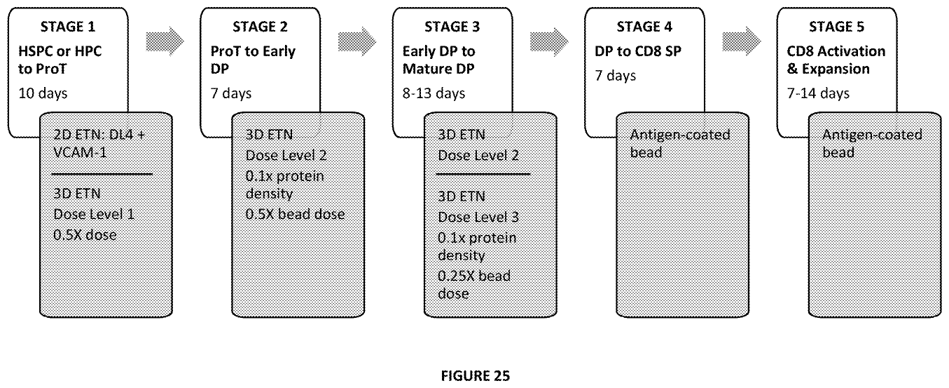

In this example, CAR-modified iPSC-derived CD34+ HPCs were differentiated into CD4−CD8+ T cells using a 5-stage protocol (protocol schematic, FIG. 25). During Stage #1, CD34 HPCs were seeded onto DL4 and VCAM-1 coated culture vessels (“2D”) or mixed with 3D ETN beads at 0.5× bead dose (2.7×10⁷ beads/mL, “3D”) in progenitor expansion medium (SFEM II+LEM, STEMCELL Technologies) and cultured for 10 days to generate CD34−CD7+CD5+ ProT cells. At this point, cells moved onto Stage #2 where ProT cells were differentiated into early DPs (CD4+CD8A+) for 7 days. CAR-modified cells were reseeded at an intermediate seeding density of 2×10⁶ cells/mL and mixed with 3D ETN at 0.1× protein density and 0.5× bead dose in progenitor maturation medium (SFEM II+LMM). At the end of stage #2, early DPs required further maturation as they do not have sufficient surface CD3/TCR expression for stimulation. During Stage #3, early DPs were allowed to mature into Late DPs (CD4+CD8A+CD8B+CD3+ TCRαβ+/−TCRγδ+/−) by re-seeding them at a higher seeding density of 6×10⁶ cells/mL, mixed with 3D ETN beads at 0.1× protein density and 0.25× bead dose in SFEM II+LMM, and cultured for an additional 8-13 days (timing may be cell line dependent). Stage #4 was started with the enrichment of Late DPs, e.g. via a CD8 Positive Selection kit (STEMCELL Technologies), followed by re-seeding in medium containing IL-21, adding CD19 antigen-coated beads (Example 1), and culturing for 7 days to allow for the DP to CD8SP conversion to occur.

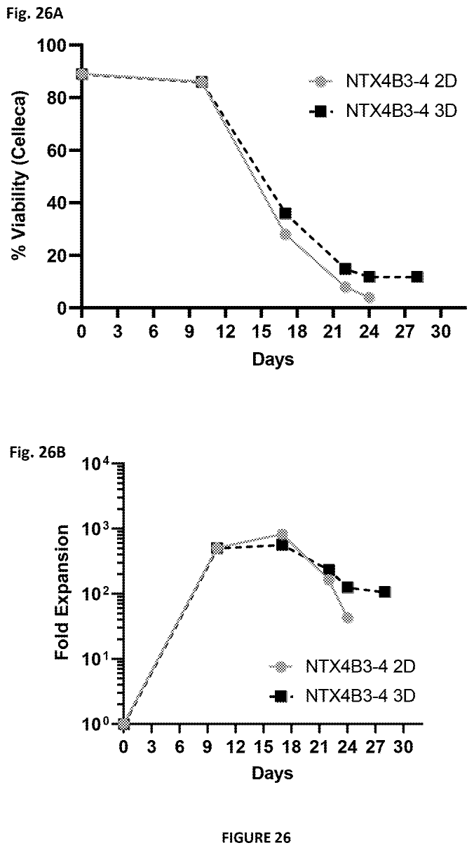

Viability and cumulative-fold expansion kinetics were evaluated during Stages 1-3 of the differentiation protocol. During the first 10 days of the process, the emerging proT cells remained highly viable and cumulative fold-expansion was at its highest (between 200-600 fold) (FIG. 10). Once proT cells entered stage #2 (Day 10-17), a substantial decrease in viability was observed with minimal increase in fold-expansion (FIG. 26). During Stage #3 (Day 17 onwards), while viability continued to decrease slightly due to improvement in DP maturity and phenotype, cells were able to sustain viabilities >10% for approximately 1 week with a minimal decrease in fold-expansion (FIG. 26A, B), which was an improvement from previous protocols.

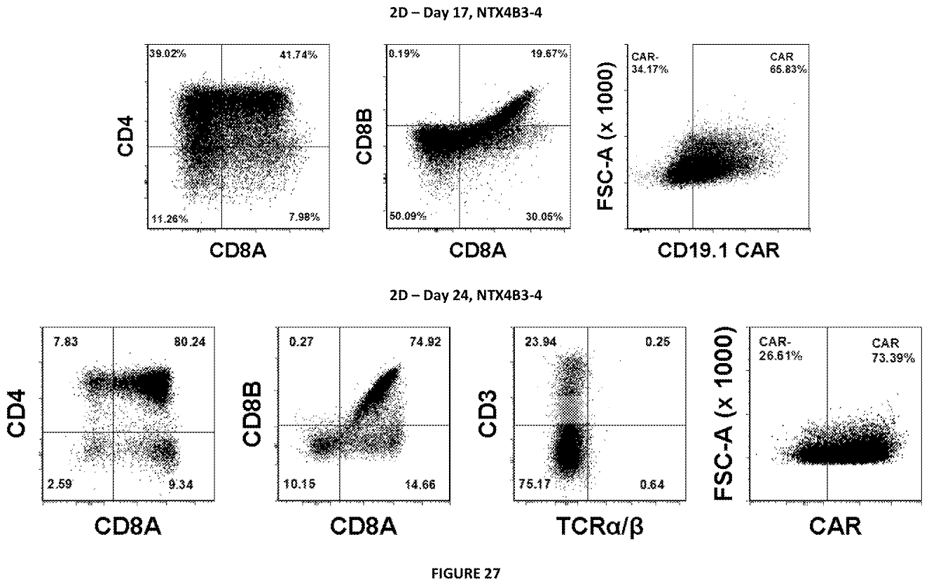

For 2D culture, Day 10 ProT cells generated in Stage #1 from CAR-modified HPCs were seeded in progenitor maturation medium (SFEM II+LMM) at 1×10⁶ cells/mL in non-tissue culture (TC)-treated plates previously coated with 1.37 μg/ml human DL4 and 1 μg/mL human VCAM-1 (intermediate 2D ETN). At day 17, sacrificial wells were harvested, and flow cytometry analysis was performed. On day 22, cultures were harvested and replated in SFEM II+LMM at 3×10⁶ cells/mL in non-TC-treated plates previously coated with 0.68 μg/ml human DL4 and 0.5 μg/mL human VCAM-1 (lower 2D ETN). At day 24, sacrificial wells were harvested, and flow cytometry analysis was performed. Lowering the 2D ETN coating and increasing seeding densities from Day 10 to Day 24 resulted in successful generation of CD4+CD8α+ Early DPs by Day 17, and effective maturation of this population as evidenced by >70% CD4+CD8α/β+, >20% surface CD3, and >70% CAR+ expression at Day 24 (FIG. 27).

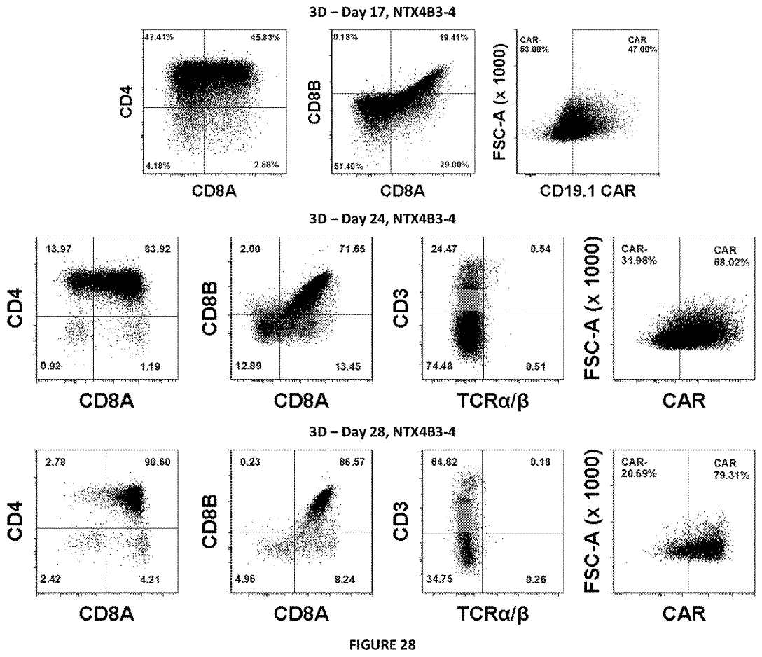

For 3D culture, day 10 ProT cells generated in Stage #1 from CAR-modified HPCs were seeded in SFEM II+LMM at 2×10⁶ cells/mL, rested for 2-4 hrs at 37° C., mixed with 0.5× bead dose of 0.1× protein density 3D ETN beads, and cultured for 7 days. At day 17, cells were harvested, flow cytometry analysis performed, and the remaining cells were reseeded in SFEM II+LMM at 5×10⁶ cells/mL, rested for 2-4 hrs at 37° C., mixed with 0.25× bead dose of 0.1× protein density 3D ETN beads, and cultured for an additional 11 days. At day 24, sacrificial wells were harvested, and flow cytometry analysis was performed. In comparison with the 2D process, the 3D protocol improved CD4+ ISP to CD4+CD8A+ DP conversion, resulting in an earlier emergence of CD4+CD8α/β+ Late DPs by Day 17 (FIG. 28, top row). While the cultures were predominantly CD4+CD8α/β+ DPs at Day 24, these Late DPs were more immature than the ones generated through the 2D protocol due to lower CD1a+ expression at this timepoint (FIG. 28, middle row). Extending the cultures for an additional 4 days resulted in improved surface CD3, CD1a, and CAR expression (FIG. 28, bottom row).

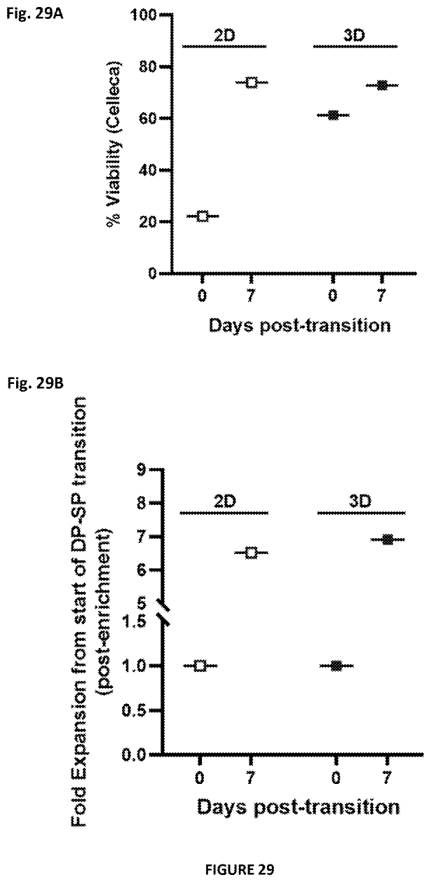

Further, late DPs were generated using either 2D ETN or 3D ETN beads. Enriched Late DPs from CAR-modified lines were seeded onto Stage #4 conditions at 1×10⁶ cells/mL and stimulated for 7 days with CD19 antigen-coated beads (Example 1) at a 1:1 bead:cell ratio. The enriched DPs generated using the 3D protocol had a higher viability than those generated using the 2D protocol (FIG. 29A). An increase in both cell number and viability was observed over time for the CAR-modified line with the CD19 bead transition protocol (FIG. 29B).

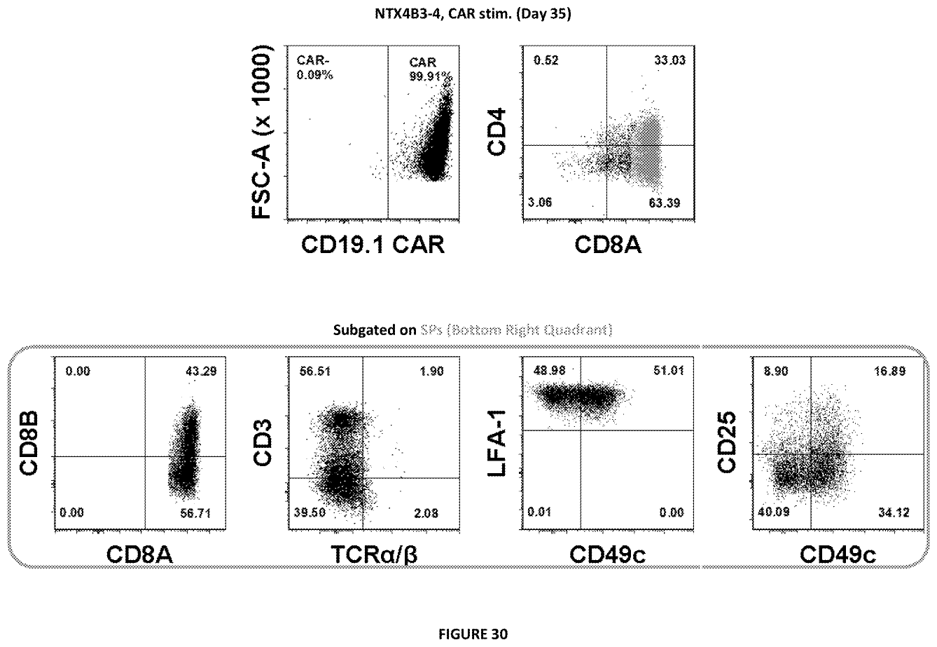

Enriched Late DPs from CAR-modified lines were seeded onto Stage #4 conditions at 1×10⁶ cells/mL and stimulated for 7 days with CD19 antigen-coated beads at a 1:1 bead:cell ratio. At the end of this stage (Day 35), cultures were harvested and stained for selected markers of T cell maturation, activation, and surface CD3 expression. Conversion with CD19 antigen-coated beads resulted in the effective generation of high mean fluorescence intensity (MFI) CAR+ CD8SPs (FIG. 30, top row). For the sub-gated CD8+ populations (FIG. 30, top row, gray overlay), these cells had intermediate levels of CD8α/β (43%), moderate CD3 expression (56%), as well as key integrins such as LFA-1 and CD49c (VLA-3) (FIG. 30, bottom row, subgated on CD8+). Expression of the activation marker CD25 was low (56%, bottom right panel).

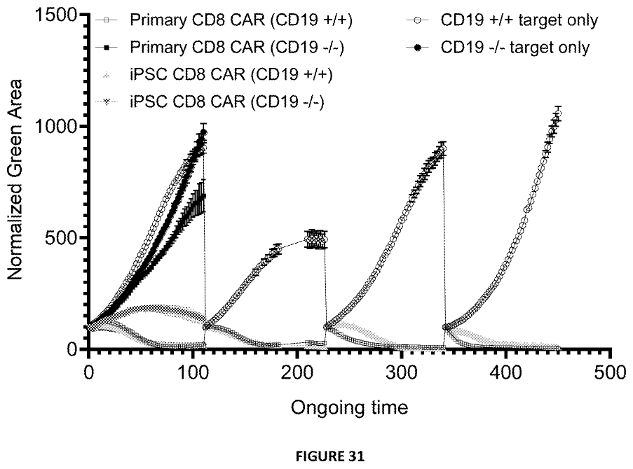

At the end of CD8SP stage (Day 35), cultures were harvested and cultured with target cells with and without CD19 expression. A serial restimulation assay was used to measure cytotoxic activity using an Incucyte®-based assay (Sartorius), with GFP-expressing CD19+ cells as target cells (n=3 technical replicates). T cells were co-cultured at a 2:1 E:T every 5 days with target cells and exogenous cytokine support. Target clearance was measured by GFP surface area reduction. CD8SP cells generated with CD19 beads had some degree of non-specific activity during the first stimulation (FIG. 31, dark gray inverted triangles) potentially owing to being activated during the DP-SP stage of differentiation. iPSC derived CD8SPs were able to serially engage target cells over 4 rounds of stimulation (FIG. 31).

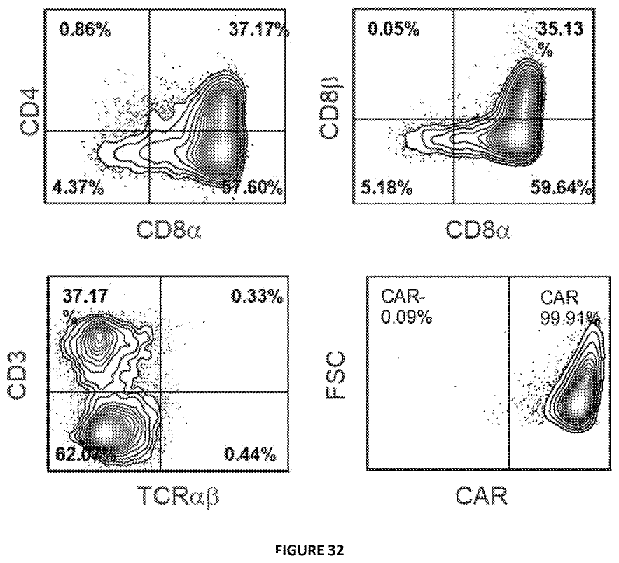

The phenotype of CD8SPs generated with the CD19 antigen-coated beads was evaluated using flow cytometry. CD8SPs were a mixture of CD8αα+ and CD8αβ+ cells with high CAR expression (FIG. 32). Some CD4 expression was still present post-SP transition (FIG. 32). Despite knockout of TRAC in iPSC cell banks, generated DPs and CD8SPs expressed surface CD3, but not TCRαβ (FIG. 32).

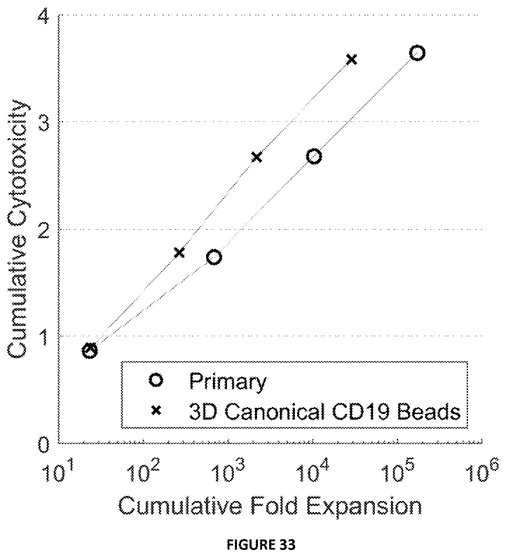

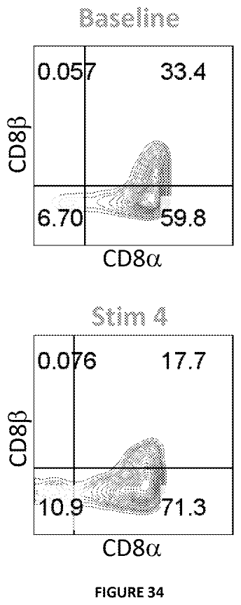

The in vitro function of iPSC-derived CD4−CD8+ cells generated with the CD19 antigen beads in the serial restimulation assay described above was compared to primary CAR-T cells by plotting cumulative cytotoxicity versus cumulative fold expansion. Comparable or higher cytotoxicity was observed for iPSC-derived cells compared to primary CAR-T cells, with primary cells exhibiting greater cumulative fold expansion (FIG. 33). For iPSC-derived cells, CD8 αβ expression was retained after 4 rounds of stimulation (FIG. 34).

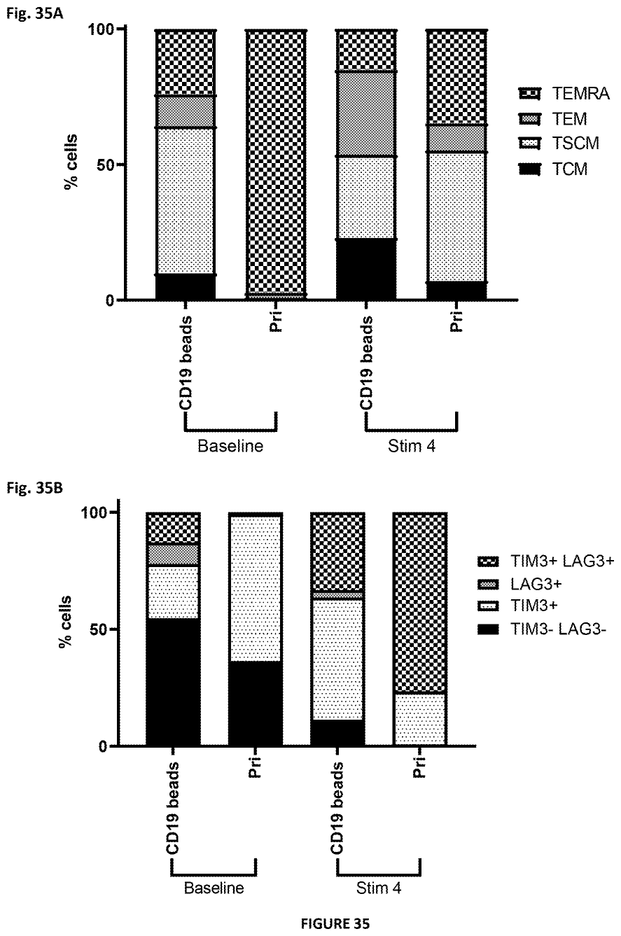

The expression of various memory markers was assessed at baseline and end of the 4^{th} round of stimulation using flow cytometry. Cells were assigned into various memory subsets based on the following expression profile: T stem cell memory (TSCM), CD62L+CD45RA+CD95+; T central memory (TCM), CD62L+CD45RA−CD45RO+; T effector memory (TEM), CD62L−CD45RA−CD45RO+; and Terminally differentiated effector memory cells re-expressing CD45RA (TEMRA), CD62L−CD45RA+CD45RO+. At baseline, a majority of primary CAR-T cells were TEMRA cells, while iPSC-derived cells included TCM, TSCM, TEM, and TEMRA phenotypes (FIG. 35A). After 4 rounds of stimulation, a greater percentage of iPSC-derived cells exhibited a TEM phenotype, and both iPSC-derived and primary cells included TSCM and TCM phenotypes (FIG. 35A).

In addition, the expression of exhaustion markers TIM3 and LAG3 before and after 4 rounds of stimulation with target cells was assessed for iPSC derived CD4−CD8+ cells generated with the CD19 antigen beads and primary CAR-T cells. Primary CAR-T cells were >60% TIM3+ at baseline, and highly TIM3+LAG3+ following target cell exposure (FIG. 35B). In contrast, iPSC derived cells had low expression of exhaustion markers at baseline, with a moderate increase in TIM3 expression following target cell exposure (FIG. 35B).

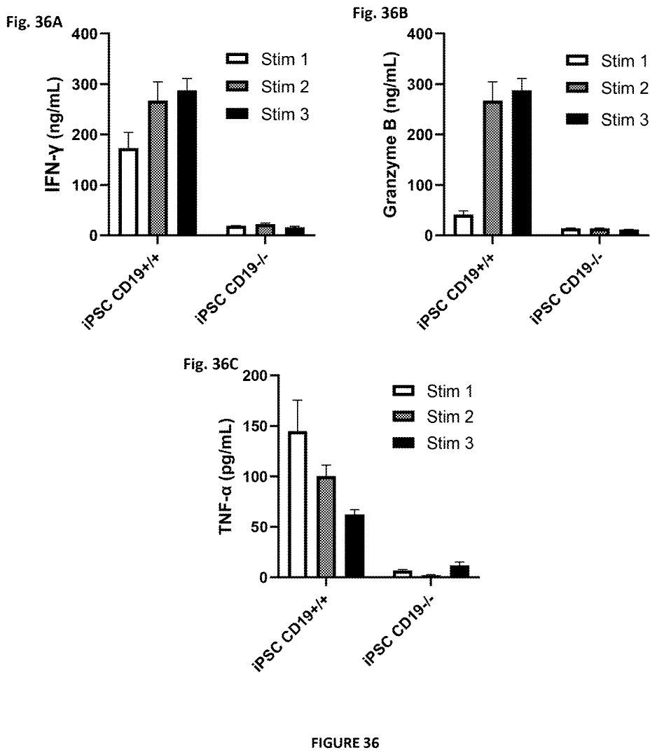

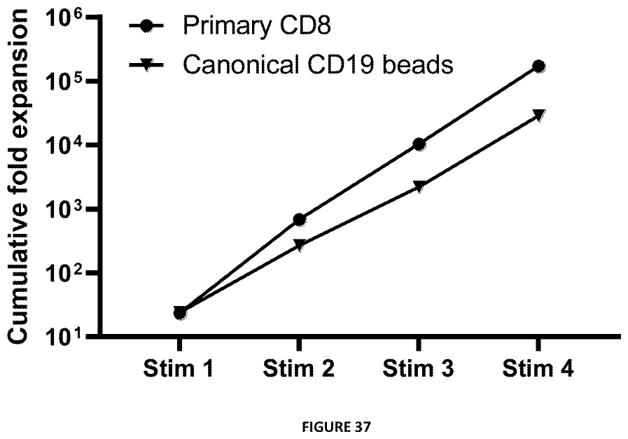

Further, cytokine secretion of iPSC derived cells was assessed. Supernatants were collected 24-36 hours after initiation of each stimulation in a long-term serial killing assay in the presence of CD19+/+ or CD19−/− target cells, and the cytokines IFNγ (FIG. 36A), Granzyme B (FIG. 36B) and TNFα (FIG. 36C) were quantified using 1300 MESO QuickPlex SQ 120MM. Cytokine secretion was found to be antigen-dependent (FIG. 36). Expansion of iPSC derived cells generated with CD19 antigen beads also demonstrated expansion over the 4 rounds of stimulation with target cells, similar to primary CD8+ CAR-T cells (FIG. 37).

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all art recited herein are incorporated herein by reference in their entirety.

### REFERENCES

1. Baulu et al. TCR-engineered T cell therapy in solid tumors: State of the art and perspectives. Science Advances eadf3700 (2023). Doi: 10.1126/sciadv.adf3700
2. Drougkas et al. Comprehensive clinical evaluation of CAR-T cell immunotherapy for solid tumors: a path moving forward or a dead end?J Cancer Res Clin Oncol 149: 2709-2734 (2023). Doi: 10.1007/s00432-022-04547-4
3. Guha et al. Assessing the Future of Solid Tumor Immunotherapy. Biomedicines 10: 655 (2022). Doi: 10.3390/biomedicines10030655
4. Iriguchi et al. A clinically applicable and scalable method to regenerate T-cells from iPSCs for off-the-shelf T-cell immunotherapy. Nat. Commun. 12, 430 (2021). Doi: 10.1038/s41467-020-20658-3
5. Klesmith et al. Retargeting CD19 Chimeric Antigen Receptor T Cells via Engineered CD19-Fusion Proteins. Mol. Pharmaceutics 16(8) 3544-3558 (2019). Doi: 10.1021/acs.molpharmaceut.9b00418
6. Lobner et al. Getting CD19 Into Shape: Expression of Natively Folded “Difficult-to-Express” CD19 for Staining and Stimulation of CAR-T Cells. Front. Bioeng. Biotechnol. 8(49): 1-13 (2020). Doi: 10.3389/fbioe.2020.00049
7. Philipson et al. 4-1BB costimulation promotes CAR T cell survival through noncanonical NF-κB signaling. Sci. Signal. 2020 13: eaay8248. Doi: 10.1126/scisignal.aay8248
8. Qu et al. Tumor buster—where will the CAR-T cell therapy ‘missile’ go? Molecular Cancer 21: 201 (2022). Doi: 10.1186/s12943-022-01669-8
9. Shukla et al. Progenitor T-cell differentiation from hematopoietic stem cells using Delta-like-4 and VCAM-1. Nat. Methods. 2017 14(5): 531-538. Doi: 10.1038/nmeth.4258
10. Sun et al. Evolution of CD8+ T Cell Receptor (TCR) Engineered Therapies for the Treatment of Cancer. Cells 10: 2379 (2021) Doi: 10.3390/cells10092379
11. Trotman-Grant et al. DL4-pbeads induce T cell lineage differentiation from stem cells in a stromal cell free system. Nat. Commun. 2021 12(5023): 1-11. Doi: 10.1038/s41467
12. Want et al. T Cell Based Immunotherapy for Cancer: Approaches and Strategies. Vaccines 11: 835 (2023). Doi: 10.3390/vaccines11040835
13. Weber et al. The Emerging Landscape of Immune Cell Therapies. Cell. 2020 181(1):46-62. Doi: 10.1016/j.cell.2020.03.001
14. Wu et al. US 20180223255
15. Zuniga-Pflucker et al. WO 2019/157597

What is claimed is:

1. A method of generating CD4−CD8+ T cells, comprising:
contacting a population of CD4+CD8+ cells expressing an antigen receptor with an antigen immobilized to a substrate, wherein the antigen binds to the antigen receptor.

2. The method of claim 1, wherein the CD4+CD8+ cells are derived in vitro from hematopoietic stem/progenitor cells.

3. The method of claim 2, wherein the hematopoietic stem/progenitor cells are derived in vitro from pluripotent stem cells.

4. The method of claim 2, wherein the CD4+CD8+ cells are derived according to a method comprising:
contacting a population of hematopoietic stem/progenitor cells with an immobilized Notch signalling ligand.

5. The method of claim 4, wherein the step of contacting the population of hematopoietic stem/progenitor cells with the immobilized Notch signalling ligand further comprises:
contacting the population of hematopoietic stem/progenitor cells with the immobilized Notch signalling ligand at a first ligand concentration, thereby generating a population of progenitor T cells; and
contacting the population of progenitor T cells with the immobilized Notch signalling ligand at a second ligand concentration, thereby generating a population of CD4+CD8+ cells, wherein the second ligand concentration is less than the first ligand concentration.

6. The method of claim 5, wherein the first ligand concentration is 3.15×10¹¹ to 1.26×10¹² molecules/mL and the second ligand concentration is 3.94×10¹⁰ to 6.31×10¹¹ molecules/mL.

7. The method of claim 5, further comprising a step of enriching the CD4+CD8+ cells for CD8α or CD8β.

8. The method of claim 4, wherein the immobilized Notch signalling ligand is DL4.

9. The method of claim 1, wherein the antigen is a CD19 polypeptide and the antigen receptor binds the CD19 polypeptide.

10. The method of claim 9, wherein the CD19 polypeptide is an engineered variant of CD19.

11. The method of claim 1, wherein the antigen is a polypeptide having the sequence of SEQ ID NO: 8.

12. The method of claim 1, wherein the substrate is a particle.

13. The method of claim 12, wherein
(a) the particle is comprised of a material selected from a group consisting of polystyrene, iron oxide and gold; or
(b) the particle is comprised of polystyrene and magnetizable iron oxides.

14. (canceled)

15. The method of claim 12, wherein the antigen is covalently conjugated to the particle.

16. The method of claim 1, wherein the step of contacting the population of CD4+CD8+ cells expressing the antigen receptor with the antigen coupled to the particle is conducted at a particle:cell ratio of 1:1.

17. A population of CD4−CD8+ T cells made according to the method of claim 1.

18. The population of claim 17, wherein
(a) the population of CD4−CD8+ T cells express a chimeric antigen receptor (CAR);
(b) the population of CD4−CD8+ T cells express a chimeric antigen receptor (CAR), wherein the CAR is a CD19 CAR and the antigen is a CD19 polypeptide;
(c) the population of CD4−CD8+ T cells is derived in vitro from pluripotent stem cells;
(d) the population of CD4−CD8+ T cells is TRAC−/− and/or CD3+; and/or
(e) the population of CD4−CD8+ T cells is greater than 50% CD8αα+.

19.-23. (canceled)

24. A method of treating a disease or condition in a subject comprising:
a) generating CD4−CD8+ T cells according to claim 1; and
b) administering an effective amount of the CD4−CD8+ T cells to a subject in need thereof.

25. (canceled)

26. A cell stimulatory particle, comprising a particle and a modified antigen immobilized on a surface of the particle, wherein the modified antigen comprises one or more amino acid substitutions from a native antigen, and wherein the modified antigen has greater solubility and/or stability in vitro in comparison to the native antigen.

27. The cell stimulatory particle of claim 26, wherein
(a) the modified antigen comprises an extracellular domain of an antigen;
(b) the modified antigen is covalently conjugated to the particle;
(c) the particle is comprised of a material selected from a group consisting of polystyrene, iron oxide and gold;
(d) the particle is comprised of polystyrene and magnetizable iron oxide;
(e) the modified antigen is a CD19 antigen; and/or
(f) the modified antigen is a polypeptide having the sequence of SEQ ID NO: 8.

28.-32. (canceled)
